(19) United States
(12) Patent Application Publication
(10) Pub. No.: US 2023/0142496 A1
(43) Pub. Date: May 11, 2023

(54) STEM CELL BIOMIMETIC NANOPARTICLE THERAPEUTIC AGENTS AND USES THEREOF

(71) Applicant: North Carolina State University, Raleigh, NC (US)

(72) Inventor: Ke CHENG, Raleigh, NC (US)

(21) Appl. No.: 17/253,159

(22) PCT Filed: Jun. 18, 2019
(86) PCT No.: PCT/US19/37609

§ 371 Date: Dec. 17, 2020

Related U.S. Application Data
(60) Provisional application No. 62/686,340, filed on Jun. 18, 2018.

(51) Int. Cl. A61K 35/28; C12N 5/0775; A61K 9/50; A61K 9/48; A61P 9/00; A61P 1/16

(52) U.S. Cl. CPC A61K 35/28; C12N 5/0663; A61K 9/5068; A61K 9/4866; A61P 9/00; A61P 1/16

(57) ABSTRACT

A stem cell biomimetic microparticle and methods of manufacture are provided that comprise a core nanoparticle and an outer layer disposed on the core nanoparticle, the core nanoparticle comprising at least one stem cell-derived secreted factor or a population of stem-cell derived exosomes embedded in a biocompatible polymer core nanoparticle, and an outer layer obtained from a cell membrane. Also provided is a method of treating a pathological condition of a patient by delivering to the patient a composition comprising the stem cell biomimetic microparticle comprising at least one stem cell-derived secreted factor or population of stem cell-derived exosomes embedded in a biocompatible polymer core microparticle and an outer layer derived from red blood cell membranes or platelet membranes disposed on the biocompatible polymer core microparticle.

### CROSS-REFERENCE TO RELATED APPLICATIONS

This application claims priority to and the benefit of U.S. Provisional Application 62/686,340 titled “STEM CELL BIOMIMETIC NANOPARTICLE THERAPEUTIC AGENTS AND USES THEREOF” filed Jun. 18, 2018, the entire disclosure of which is incorporated herein by reference in its entirety.

### STATEMENT REGARDING FEDERALLY SPONSORED RESEARCH OR DEVELOPMENT

This invention was made with government support under grant numbers HL137093 and HL123920 awarded by the National Institutes of Health. The government has certain rights in the invention.

### TECHNICAL FIELD

The present disclosure is generally related to a stem cell-derived nanoparticle encapsulated with cell membranes. The present disclosure further relates to methods of treatment of a pathological condition responsive to a stem cell-derived secretome or fraction thereof.

### BACKGROUND

Acute liver failure is a critical condition characterized by global hepatocyte death and often time needs a liver transplantation. Such treatment is largely limited by donor organ shortage. Stem cell therapy offers a promising option to patients with acute liver failure. Yet, therapeutic efficacy and feasibility are hindered by delivery route and storage instability of live cell products. A nanoparticle was fabricated that carries the beneficial regenerative factors from mesenchymal stem cells and further coated it with the membranes of red blood cells to increase blood stability. Unlike un-coated nanoparticles, these particles promote liver cell proliferation in vitro and have lower internalization by macrophage cells. After intravenous delivery, these artificial stem cell-analogs are able to remain in the liver and mitigate carbon tetrachloride-induced liver failure in a mouse model, as gauged by histology and liver function test.

Acute liver failure, which follows an initial insult such as infection and toxic reagents, is a lethal condition characterized by global hepatocyte necrosis, acute deterioration of liver function and subsequent multi-organ failure (Elias E. (2006) Hepatology 43: S239-242). Orthotopic liver transplantation is currently viewed the most effective treatment option for liver failure, but its use is limited by donor availability and the requirement for lifelong immunosuppression (Starzl T. E. (2012) Nat. Med. 18: 1489-1492; Jalan et al., (2102) J. Hepatol. 57: 1336-1348). Therefore, alternative therapeutic and regenerative strategies for acute liver failure are urgently needed (Itoh & Miyajima (2014) Hepatology 59: 1617-1626).

Stem cell therapy offers an alternative strategy for treating hepatic diseases (Zhang & Wang (2013) J. Hepatol. 59: 183-185). It has been shown that mesenchymal stem cells (MSCs) or MSC-derived microvesicles are able to mitigate acute liver failure in various animal models and recently completed human trials (Li et al., (2012) Hepatology 56: 1044-1052; Shi et al., (2017) Gut 66: 955-964; Lin et al., (2017) Hepatology 66: 209-2019). However, cell therapy suffers several limitations. First, as a “live drug”, stem cell products need to be freshly prepared or carefully cryo-stored before final formulation. Moreover, due to their sizes, intravenously infused MSCs will get stuck in the lungs before they can reach the targeted organ (e.g. the liver). Portal vein injection can deliver a good amount of cells to the liver yet this method is invasive and repeated dosing is difficult. Numerous studies indicate that MSCs produce their functional benefits mainly through paracrine effects, i.e. secreted factors from MSCs promote healing and inhibit fibrosis and inflammation (Chen et al., (2017) Stem Cell Res. Ther. 8: 9). Therapeutic cell-mimicking particles had been previously fabricated by encapsulating MSC-secreted factors in a biodegradable polymer shell and tested their regenerative potential in a rodent model of heart injury (Luo et al., (2017) Circ. Res. 120: 1768-1775).

In recent years, there has been a tremendous success in the development of cell membrane-coated nanoparticles (Hu et al., (2011) Proc. Natl. Acad. Sci. U.S.A. 108: 10980-10985; Hu et al., (2015) Nature 526: 118-121). The membranes from various cell types empower nanoparticles with functionalities that have been created and perfected by the nature, such as excellent stability in the blood (Sun et al., (2016) Adv. Mater. 28: 9581-9588; Fang et al., (2014) Nano Lett. 14: 2181-2188; Hu et al., (2015) Adv. Mater. 27: 7043-7050). For instance, it has been reported that nanoparticles coated with the membranes of red blood cells (RBCs) can preserve the membrane protein complexes that are essential for long blood circulation of RBCs (Mohandas & Gallagher (2008) Blood 112: 3939-3948), prolonging the blood half-life of those nanoparticles and amplifying their therapeutic effects (Piao et al., (2014) ACS Nano 8: 10414-10425).

Cardiovascular disease is the leading cause of mortality globally, accounting for over 17.3 million deaths each year. Coronary heart disease, including myocardial infarction (commonly known as heart attack), angina (chest pain), and cardiac arrest, contributes to at least 50% of death (Townsend et al., (2016) Eur. Heart J. 37: 3232). Coronary artery obstruction produces myocardial ischemia, which is usually treated with reperfusion therapy. While reperfusion of ischemic tissue is vital for survival, it also initiates myocardial ischemia/reperfusion (I/R) injury comprising oxidative damage, cell death, and a profound inflammatory immune response, which currently lacks an effective clinical therapy (Chouchani et al., (2014) Nature 515: 431).

In the past decades, the potential of using stem cells, including mesenchymal stem cells (MSCs), cardiac stem cells (CSCs), and induced pluripotent stem cells (iPSCs), for cardiac regenerative therapy has generated immense interest (Bolli et al., (2017) Nat. Rev. Cardiol. 14: 257). Numerous studies indicate that stem cells exert their functional benefits mainly through paracrine effects, i.e., secreted factors from stem cells promote cardiac regeneration and inhibit fibrosis and inflammation. However, stem cell therapy suffers from several limitations, such as low cellular retention and survival in the ischemic myocardium, cryopreservation and transportation issues, and easy entrapment in the lung during intravenous delivery due to their sizes (Lin & Pu, (2014) Sci. Transl. Med. 6: 239rv1; Fischer et al., (2009) Stem Cells Dev. 18: 683).

Nanotechnology holds great promise to revolutionize cardiovascular therapy. In recent years, the development of nanoparticles for active targeting the heart after ischemic injury is receiving increasing attention (Dviret al., (2011) Nano Lett. 11: 4411; Paulis et al., (2012) J. Control. Release 162: 276; Nguyen et al., (2015) Adv. Mater. 27: 5547). The enhanced permeability and retention (EPR) effect of the leaky vasculature in the infarcted heart has been commonly used for designing targeted nanoparticles, although these nanoparticles are limited by rapid clearance within a few hours to days (Paulis et al., (2012) J. Control. Release 162: 276). A recently reported matrix metalloproteinase (MMP)-responsive nanoparticle showed successful retention in the infarcted heart for up to 28 days (Nguyen et al., (2015) Adv. Mater. 27: 5547). Yet, the therapeutic efficacy of these nanoparticle systems in protecting the heart from I/R injury still remains elusive.

Recently, cell membrane-coated nanoparticles have emerged as a novel platform that can successfully combine the functionalities of various types of cells (36 Wang et al., (2016) Adv. Funct. Mater. 26: 1628; Anselmo et al., (2014) ACS Nano 8: 11243; Hu et al., (2016) Adv. Mater. 28: 9573; Dehaini et al., (2017) Adv. Mater. 29: 1606; Hu et al., (2015) Adv. Mater. 27: 7043; Hickman et al., Adv. Mater. 30: 1700859). Fusing platelet-derived nanovesicles onto the surface of CSCs boosts the infarct-targeting ability and functional outcomes of CSCs in rats and pigs with myocardial infarction (Tang et al., (2018) Nat. Biomed. Eng. 2: 17). Therapeutic cell-mimicking particles were fabricated by encasing CSC- or MSC-secreted factors in a biodegradable polymer shell coated with cell membranes and tested their regenerative potential in a rodent model of heart injury (Luo et al., (2017) Circ. Res. 120: 1768; Tang et al., (2017) Nat. Commun. 8: 13724). Prostaglandin E2 (PGE₂) is an FDA-approved medication (known as dinoprostone) that participates in many biological pathways. PGE₂ exerts its physiologic effects via four subtypes of receptors (EPs), i.e., EP1, EP2, EP3, and EP4, among which EP2, EP3, and EP4 are overexpressed on the surface of cardiomyocytes following I/R injury (Kim et al., (2008) Biomaterials 29: 4439; Zhang et al., (2015) Science 348: aaa2340). Recent studies have identified PGE₂ as an important signaling molecule that activates endogenous stem/progenitor cells for cardiac repair post-ischemic injury (Hsueh et al., (2014) EMBO Mol. Med. 6: 496).

### SUMMARY

Briefly described, one aspect of the disclosure encompasses embodiments of a stem cell biomimetic microparticle comprising a core nanoparticle and an outer layer disposed on the core nanoparticle, the core nanoparticle comprising at least one stem cell-derived secreted factor or a population of stem-cell derived exosomes embedded in a biocompatible polymer core nanoparticle, and wherein the outer layer is obtained from a cell membrane.

In some embodiments of this aspect of the disclosure, the stem cell can be a mesenchymal stem cell or a cardiac stem cell.

In some embodiments of this aspect of the disclosure, the cell membrane can be a red blood cell outer membrane or a stem cell outer membrane.

In some embodiments of this aspect of the disclosure, the cell membrane can be isolated from platelets.

In some embodiments of this aspect of the disclosure, the biocompatible polymer core nanoparticle can consist of a single species of polymer, a plurality of biocompatible polymer species, a block copolymer, or a plurality of polymer species, and wherein the polymer or polymers of the polymer core can be cross-linked.

In some embodiments of this aspect of the disclosure, the biocompatible polymer core nanoparticle can be biodegradable.

In some embodiments of this aspect of the disclosure, the biocompatible polymer core can comprise poly(lactic-co-glycolic acid) (PLGA).

In some embodiments of this aspect of the disclosure, the cell membrane can comprise a ligand attached thereto, wherein the ligand can specifically bind to a target cell or tissue.

In some embodiments of this aspect of the disclosure, the cell membrane can be isolated from platelets and the ligand is prostaglandin E2 (PGE₂).

In some embodiments of this aspect of the disclosure, the at least one stem cell-derived secreted factor or exosome population and the cell membrane layer can be from the same individual.

In some embodiments of this aspect of the disclosure, the at least one stem cell-derived secreted factor or exosome population and the cell membrane layer can be from different individuals.

In some embodiments of this aspect of the disclosure, the at least one stem cell-derived secreted factor can be from a stem cell-conditioned culture medium.

In some embodiments of this aspect of the disclosure, the at least one stem cell-derived secreted factor can be from a mesenchymal or cardiac stem cell-conditioned culture medium.

In some embodiments of this aspect of the disclosure, the at least one stem cell-derived secreted factor can be an isolated mesenchymal or cardiac stem cell-derived secreted factor.

In some embodiments of this aspect of the disclosure, the population of exosomes can be isolated from a medium conditioned by culturing therein a population of stem cells.

In some embodiments of this aspect of the disclosure, the population of exosomes can be isolated from a medium conditioned by culturing therein a population of stem cells.

In some embodiments of this aspect of the disclosure, the population of exosomes can be from a mesenchymal or cardiac stem cell-conditioned culture medium.

In some embodiments of this aspect of the disclosure, the microparticle can be suspended in a pharmaceutically acceptable carrier.

Another aspect of the disclosure encompasses embodiments of a method of generating a biomimetic microparticle, said method comprising the steps of: (a) admixing an aqueous solution comprising at least one stem cell-derived secreted factor or population of stem cell-derived exosomes with an organic phase having a polymerizable monomer dissolved therein; (b) emulsifying the admixture from step (a); (c) admixing the emulsion from step (b) with an aqueous solution of polyvinyl alcohol and allowing the organic phase to evaporate, thereby generating polymer nanoparticles having the at least one stem cell-derived secreted factor embedded therein; (d) obtaining an isolated cell membrane or fragments thereof; and (e) generating cell membrane-coated biomimetic microparticles by mixing the polymer nanoparticles from step (c) with the suspension of isolated red blood cell membrane or fragments thereof.

In some embodiments of this aspect of the disclosure, the aqueous solution comprising the at least one stem cell-derived secreted factor can be a stem cell-conditioned culture medium or an aqueous solution of at least one isolated stem cell secreted factor.

In some embodiments of this aspect of the disclosure, the aqueous solution can be a mesenchymal stem cell- or a cardiac stem cell-conditioned culture medium.

In some embodiments of this aspect of the disclosure, the first polymerizable monomer polymer can be poly(lactic-co-glycolic acid) (PLGA).

In some embodiments of this aspect of the disclosure, the step (c) can further comprise lyophilizing the polymer microparticles.

Yet another aspect of the disclosure encompasses embodiments of a method of treating a pathological condition of a patient by delivering to the patient in need thereof a pharmaceutically acceptable composition comprising a stem cell biomimetic microparticle, wherein the stem cell biomimetic microparticle comprises at least one stem cell-derived secreted factor or population of stem cell-derived exosomes embedded in a biocompatible polymer core microparticle and an outer layer derived from red blood cell membranes or platelet membranes disposed on the biocompatible polymer core microparticle.

In some embodiments of this aspect of the disclosure, the pathological condition of the patient can be a disease of the liver.

In some embodiments of this aspect of the disclosure, the pathological condition of the patient can be a cardiovascular disease or injury.

In some embodiments of this aspect of the disclosure, the biocompatible polymer core of the microparticle can consist of a single species of polymer, a plurality of polymer species, a block copolymer, or a plurality of polymer species, and wherein the polymers of the polymer core can be cross-linked.

In some embodiments of this aspect of the disclosure, the polymer core of the microparticle can be biodegradable.

In some embodiments of this aspect of the disclosure, the polymer core can comprise poly(lactic-co-glycolic acid) (PLGA).

In some embodiments of this aspect of the disclosure, the at least one stem cell-derived secreted factor and the cell membrane fragment or fragments can be from the same individual.

In some embodiments of this aspect of the disclosure, the at least one stem cell-derived secreted factor and the cell membrane fragment or fragments can be from different individuals.

In some embodiments of this aspect of the disclosure, the at least one stem cell-derived secreted factor can be from a mesenchymal stem cell- or cardiac stem-cell-conditioned culture medium.

In some embodiments of this aspect of the disclosure, the at least one stem cell-derived secreted factor can be an isolated secreted factor.

In some embodiments of this aspect of the disclosure, the pharmaceutically acceptable composition can be formulated for delivery directly into a target tissue of a subject animal or human or for local or systemic administration to a subject animal or human.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A and 1B illustrate the characterization MSCs and fabrication of MRINs.

FIG. 1A is a schematic illustration showing the fabrication of MRINs involves three steps: preparation of membrane vesicles from RBCs; preparation of nanoparticles (NPs) from MSC-conditioned media, and cloaking of RBC membranes on NPs to make MRINs.

FIG. 1B illustrates a flow cytometry analysis of common MSC markers such as CD105, CD90, CD45, CD31 and CD34 (n=3 for each marker).

FIGS. 2A-2L illustrate the physiochemical and biological properties of MRINs.

FIGS. 2A and 2B illustrate TEM images of a NP and a MRIN. The nanoparticles were negatively stained with uranyl acetate and subsequently visualized with TEM.

FIG. 2C illustrates diameters (nm) of NPs and MRINs measured by NanoSight® (n=4).

FIG. 2D illustrates the zeta potential (mV) of NPs and MRINs (n=3).

FIG. 2E illustrates images of SDS-PAGE gels examining protein contents of MRIN, which have similar protein bands as those observed from RBC membranes.

FIG. 2F illustrates size distribution of MRIN measured by NanoSight. The average size is approximately 200 nm.

FIG. 2G illustrates a TEM image of a MRIN after freeze/thaw, showing the RBC coat is still intact.

FIG. 2H illustrates diameters (nm) of MRIN before and after freeze/thaw (n=4).

FIG. 2I illustrates Size change of MRINs after storage at room temperature (n=3 for each time point).

FIG. 2J illustrates zeta potential (mV) of MRIN before and after freeze/thaw (n=3).

FIG. 2K illustrates blood retentions of NPs and MRINs over a span of 48 hrs. (n=3). (2L) Quantitative analyses on the releases of stromal cell-derived factor-1 (SDF-1), insulin-like growth factor-1 (IGF-1) and hepatocyte growth factor (HGF) from NPs and MRINs over time. n.s. indicated P>0.05. Comparisons between any two groups were performed using two-tailed unpaired Student's t-test. Scale bars, 100 nm. All data are expressed the Means±SD.

FIGS. 3A-3L illustrate the effects of MRINs on liver, lung, and M1 macrophage cells.

FIGS. 3A-3C illustrate the morphology of THLE-2 Cells, HSC-T6 cells and mouse lung cells indicated by white light microscopy. Scale bars, 50 μm.

FIG. 3D illustrates the percentages of proliferating THLE-2 cells under various treatments (n=6 for each group).

FIGS. 3E and 3F illustrate the proliferation of HSC-T6 cells and mouse lung cells under various concentrations of MRINs (n=6 for each group).

FIGS. 3G and 3I illustrate the macrophage uptake of NPs and MRINs after 3 hrs of co-incubation. Scale bars, 10 μm.

FIG. 3J illustrates the quantitative analysis of the percentages of macrophages with particle endocytosis (n=4 for each group).

FIG. 3K illustrates a representative confocal microscopy image showing the minimal internalization of MRINs by liver macrophages (stained with CD45). Scar bar, 50 μm.

FIG. 3L illustrates a representative confocal microscopy image showing the location of MRINs relative to liver cells (stained with WGA). Scar bar, 20 μm. * indicates P<0.05; ** indicates P<0.01; *** indicates P<0.0001; n.s. indicated P>0.05. Student'st-test for comparison between 2 groups and one-way ANOVA for comparison among 3 and more groups. All data are expressed the Means±SD.

FIGS. 4A-4F illustrate biodistrbution of MRINs and effects on animal survival.

FIG. 4A illustrates a schematic showing the animal study design.

FIG. 4B illustrates a tabular summary of study groups.

FIG. 4C illustrates liver histology after CCL4 treatment. H & E staining showing confluent necrosis of the hepatic lobules in model mice but not in normal mice.

FIG. 4D illustrates survival rates of animal in various groups (n=10 animals per group).

FIG. 4E illustrates the biodistributions of NPs and MRINs after systemic administration in mice with acute liver failure. Representative ex vivo fluorescent imaging of mouse organs (heart, lungs, liver, spleen and kidneys) at 6, 12 and 24 hrs post intravenous NP and MRIN injections.

FIG. 4F illustrates quantitative analysis of fluorescent intensities (n=3 animals per group). # indicates P<0.01 when compared to NP group. Student's t-test for comparison between 2 groups and one-way ANOVA for comparison among 3 and more groups. All data are expressed as the means±SD.

FIGS. 5A-5G illustrate that MRIN therapy improves liver function and reduces systemic inflammation.

FIGS. 5A and 5B illustrate serum levels of ALT and AST were measured 24 h after CCL4 injection (baseline) and 3 or 7 days after treatment (n=3 for each group). # indicates P<0.05 when compared with NP group; & indicates P<0.05 when compared with CM group.

FIGS. 5C and 5D illustrate serum levels of BUN and Cr measured 2 weeks after therapy (n=1 for PBS group and n=3 for other groups).

FIGS. 5E-5G illustrate serum levels of IL-1β, IL-6, and TNF-α measured 2 weeks after therapy (n=1 for PBS group and n=3 for other groups). * indicates P<0.05; ** indicates P<0.01; n.s. indicated P>0.05. Student'st-test for comparison between 2 groups and one-way ANOVA for comparison among 3 and more groups. All data are presented as mean±SD.

FIGS. 6A-6E illustrate that MRIN therapy promotes liver cell regeneration and inhibits cell death.

FIG. 6A illustrates H & E staining of liver sections from different groups. Scale bars, 50 μm.

FIG. 6B illustrates a representative fluorescent micrographs showing Ki67-positive cells (red nuclei). Scale bars, 50 μm.

FIG. 6C illustrates a representative fluorescent micrographs showing TUNEL-positive cells (red nuclei). Scale bars, 50 μm.

FIGS. 6D and 6E illustrate the quantitation of proliferative and apoptotic cells (n=3 for each group). * indicates P<0.05; *** indicated P<0.001. Student's t-test for comparison between 2 groups and one-way ANOVA for comparison among 3 and more groups. All data are expressed the Means±SD.

FIGS. 7A-7C illustrate the biodistribution of MRINs in normal mice.

FIG. 7A illustrates a representative ex vivo fluorescent imaging of major organs (heart, lungs, liver, spleen and kidneys) 6, 12, and 24 hrs post intravenous NP and MRIN injections.

FIG. 7B illustrates a quantitative analysis of fluorescent intensities (n=3 animals per group).

FIG. 7C illustrates a comparison of MRIN biodistribution in normal and liver failure mice (n=3 animals per group). * indicates P<0.05 when compared to NP group; # indicates P<0.01 when compared to NP group; n.s. indicates P>0.05. Student's t-test for comparison between 2 groups and one way ANOVA for comparison among 3 and more groups. All data are expressed the Means±SD.

FIGS. 8A-8I illustrate the fabrication and characterization of PINCs.

FIG. 8A illustrates a schematic illustration of the fabrication process of PINCs. The therapeutic effects of PINC injection were tested in mice with myocardial I/R injury.

FIG. 8B illustrates a TEM image showing the ultrastructure of CS-PGE₂-PINC.

FIG. 8C illustrates a size distribution of CS-PGE₂-PINC measured by DLS.

FIG. 8D illustrates zeta potentials of CS-PGE₂-PINC and NC.

FIG. 8E illustrates particle sizes of bare NC and CS-PGE₂-PINC over 2 weeks in PBS.

FIG. 8F illustrates a TEM image showing the ultrastructure of CS-PGE₂-PINC after freeze-thawing.

FIGS. 8G and 8H illustrate a comparison of particle size (FIG. 8G) and zeta potential (FIG. 8H) of CS-PGE₂-PINC before and after freeze-thawing.

FIG. 8I illustrates in vitro stability of NC, CS-PINC, and CS-PGE2-PINC before and after incubation in 50% fetal bovine serum. Scale bars, 100 nm. All data are mean±s.d. * indicates p<0.05, ** indicates p<0.01. Comparisons among more than two groups were performed using one-way ANOVA followed by post hoc Bonferroni test.

FIGS. 9A-9K illustrate in vitro bioactivity of PINCs.

FIGS. 9A-9C illustrate quantitative analysis of the releases of SDF-1, HGF, VEGF from NC and CS-PGE₂-PINC over 2 weeks.

FIG. 9D illustrates a protein content visualization of platelet membrane (PM), PGE₂-PINC, CS-PINC, and CS-PGE₂-PINC run on SDS-PAGE at equivalent protein concentrations.

FIG. 9E illustrates a collagen-coated 4-well slides seeded with human umbilical vein endothelial cells (HUVECs) were incubated with CS-PGE₂-PINCs for 60 s, followed by fluorescence microscopy showing selective CS-PGE₂-PINC adherence to exposed collagen versus endothelial surfaces.

FIG. 9F illustrates a quantification of CS-PGE₂-PINC in endothelial- and collagen-covered surface, respectively.

FIG. 9G illustrates a cytocompatibility of PINCs at various concentrations.

FIG. 9H illustrates the proliferation of H9c2 cells over time in the presence of different PINCs.

FIGS. 9I and 9J illustrates representative confocal image showing the internalization of CS-PINC (FIG. 9I) and CS-PGE₂-PINC (FIG. 9J) by NRCMs.

FIG. 9K illustrates a quantitative analysis of the percentage of NRCMs with different nanoparticle endocytosis. FIG. 9K illustrates a quantitative analysis of NRCM contractility when co-cultured with different PINCs.

Scale bars, (FIG. 9E) 20 μm; (FIGS. 9I and 9J) 50 μm. All data are mean±s.d. Comparisons between any two groups were performed using two-tailed unpaired Student's t-test. Comparisons among more than two groups were performed using one-way ANOVA followed by post hoc Bonferroni test. * indicates p<0.05, ** indicates p<0.01, *** indicates p<0.001.

FIGS. 10A-10E illustrate biodistribution and in vivo bioactivity of PINCs.

FIG. 10A illustrates a schematic showing the animal study design.

FIG. 10B illustrates biodistributions of CS-PGE₂-PINCs and NCs after intravenous delivery in mice with myocardial I/R injury. Representative ex vivo fluorescent imaging of mouse organs (heart, lung, liver, kidney, and spleen) at 14 days post-intravenous injections of CS-PGE₂-PINCs and NCs.

FIG. 10C illustrates a quantitative analysis of fluorescent intensities (n=3 animals per group).

FIG. 10D illustrates representative images showing cycling cardiomyocytes (yellow arrowheads) as indicated by α-SA and Ki67 double-positive staining in the peri-infarct regions of the hearts treated with CS-PINCs, PGE₂-PINCs, and CS-PGE₂-PINCs at week 4.

FIG. 10E illustrates a quantification of Ki67-positive cardiomyocytes at week 4 in the saline control (n=5), CS-PINC (n=6), PGE₂-PINC (n=6), and CS-PGE₂-PINC (n=6) groups.

Scale bars, 50 μm. All data are mean±s.d. Comparisons between any two groups were performed using two-tailed unpaired Student's t-test. Comparisons among more than two groups were performed using one-way ANOVA followed by post hoc Bonferroni test. * indicates p<0.05, ** indicates p<0.01.

FIGS. 11A-11D illustrate in vivo mitotic activities of cardiomyocytes.

FIGS. 11A and 11B illustrate the visualization of phosphohistone H3 phosphorylation in cardiomyocytes (yellow arrowheads) in the peri-infarct regions of saline control-, CS-PINC-, PGE₂-PINC-, and CS-PGE₂-PINC-treated hearts at week 4. Representative images in FIG. 11A indicate cells that are in late G2/mitosis phase; the square highlights the localization of pH 3 (arrowheads) in the nuclei of cycling cardiomyocytes). Quantification in FIG. 11B shows pH 3-positive cardiomyocytes at week 4 in the saline control (n=5), CS-PINC (n=6), PGE₂-PINC (n=6), and CS-PGE₂-PINC (n=6) groups.

FIGS. 11C and 11D illustrate the visualization of AURKB in cardiomyocytes (arrowheads) in the peri-infarct regions of saline control-, CS-PINC-, PGE₂-PINC-, and CS-PGE₂-PINC-treated hearts at week 4. Representative images in FIG. 11C indicate DAPI staining nuclei; α-sarcomeric actinin staining cardiomyocytes; AURKB marking the cells in karyokinesis and cytokinesis; the square highlights the localization of AURKB in midbodies (arrowheads). Quantification in FIG. 11D shows AURKB-positive cardiomyocytes at week 4 in the saline control (n=5), CS-PINC (n=6), PGE₂-PINC (n=6), and CS-PGE₂-PINC (n=6) groups. Scale bars, 20 μm. All data are mean±s.d. Comparisons among more than two groups were performed using one-way ANOVA followed by post hoc Bonferroni test. * indicates p<0.05; ** indicates p<0.01; *** indicates p<0.001.

FIGS. 12A-12G illustrate functional benefits of PINC therapy in mice with myocardial I/R injury.

FIG. 12A illustrates representative Masson's trichrome sections showing scar tissue (blue) and viable myocardium (red) in the hearts 4 weeks after treatment with saline (n=5), CS-PINCs (n=6), PGE₂-PINCs (n=6), and CS-PGE₂-PINCs (n=6), respectively.

FIGS. 12B and 12C illustrate quantitative analyses of viable myocardium (FIG. 12B) and scar size (FIG. 12C) from the Masson's trichrome images.

FIGS. 12D and 12E) illustrate left ventricular end-diastolic (FIG. 12D) and end-systolic (FIG. 12E) volumes (LVEDV and LVESV) measured by echocardiography at 4 weeks after I/R in mice treated with saline, CS-PINCs, PGE₂-PINCs, and CS-PGE₂-PINCs, respectively.

FIG. 12F illustrates left ventricular ejection fraction (LVEF) measured by echocardiography at baseline (4 h post-I/R) and 4 weeks afterward in the saline, CS-PINC, PGE₂-PINC, and CS-PGE₂-PINC groups. Scale bar, 2 mm. All data are mean±s.d. * indicates p<0.05; ** indicates p<0.01; *** indicates p<0.001.

FIG. 12G illustrates that treatment effects were assessed by the change in LVEF over the 4-week time course relative to baseline. # indicates p<0.05 when compared with saline control group; t indicates p<0.05 when compared with any other groups.

FIGS. 13A-13F illustrate PINC injection promotes endogenous repair in the infarcted heart.

FIGS. 13A-13C illustrate representative images showing Nkx2.5 and c-kit double-positive cells, CD34-positive cells, and vWF-positive capillaries in the infarcted hearts 4 weeks after saline (n=5), CS-PINC (n=6), PGE2-PINC (n=6), or CS-PGE₂-PINC (n=6) treatment. Arrowheads indicate the positively stained cells.

FIGS. 13D-13F illustrate quantification of the number of Nkx2.5 and c-kit double-positive cells (FIG. 13D), CD34-positive cells (FIG. 13E), and vWF-positive capillary density (FIG. 13F) in the infarcted hearts 4 weeks after saline (n=5), CS-PINC (n=6), PGE2-PINC (n=6), or CS-PGE₂-PINC (n=6) treatment. Scale bars, (A, B) 20 μm; (C) 100 μm. Comparisons among more than two groups were performed using one-way ANOVA followed by post hoc Bonferroni test. * indicates p<0.05; ** indicates p<0.01; *** indicates p<0.001.

FIG. 14 illustrates the synthesis of the PGE₂-platelet membrane conjugates.

FIG. 15 illustrates confocal images of PGE₂-platelet membrane conjugate. The platelet membrane was labeled with Dil prior to the synthesis of PGE₂-platelet membrane conjugate. Subsequently, the Dil-labeled PGE₂-platelet membrane conjugate was incubated with FTIC-labeled PGE₂ antibody overnight before imaging. Scale bars, 10 μm.

FIGS. 16A and 16B illustrates a nanoparticle tracking analysis (NTA) of CS-PGE₂-PINCs using NanoSight.

FIG. 16A illustrates a representative particle tracking image of CS-PGE₂-PINCs

FIG. 16B illustrates size distribution as reported by NTA showing that the average size of CS-PGE₂-PINC is about 205 nm, consistent with those determined by DLS and TEM analysis.

FIG. 17 illustrates the average particle sizes of the as-prepared NCs and CS-PGE₂-PINCs in PBS (1×, pH 7.4). All data are mean±s.d. (n=3). Comparisons between any two groups were performed using two-tailed unpaired Student's t-test. * indicates p<0.05, ** indicates p<0.01, *** indicates p<0.001.

FIG. 18 illustrates the zeta potentials of platelet membrane (PM), CS-PINCs, and PGE₂-PINCs in PBS (1×, pH 7.4). All data are mean±s.d. (n=3). Comparisons among more than two groups were performed using one-way ANOVA followed by post hoc Bonferroni test. * indicates p<0.05, ** indicates p<0.01, *** indicates p<0.001.

FIG. 19 illustrates the average particle sizes of CS-PINC and PGE₂-PINC over 2 weeks in PBS (1×, pH 7.4). All data are mean±s.d. (n=3).

FIGS. 20A and 20B illustrate the particle size distribution of CS-PGE₂-PINC before freezing at −80° C. (FIG. 20A) and after thawing at room temperature 3 months later (FIG. 20B).

FIG. 21 illustrates the particle sizes of CS-PINC, PGE₂-PINC, and CS-PGE₂-PINC before lyophilization in 10 wt % sucrose and after resuspension in PBS (1×, pH 7.4). All data are mean±s.d. (n=3). Comparisons between any two groups were performed using two-tailed unpaired Student's t-test. * indicates p<0.05, ** indicates p<0.01, *** indicates p<0.001.

FIG. 22A illustrates representative images showing the binding of CS-PEG2-PINCs to the collagen-coated 4-well culture chamber slide surface relative to the non-coated surface.

FIGS. 22B illustrates quantification of the numbers of different nanoparticles binding to the collagen-coated and non-coated surfaces, respectively. All data are mean±s.d. (n=3). Comparisons between any two groups were performed using two-tailed unpaired Student's t-test. Comparisons among more than two groups were performed using one-way ANOVA followed by post hoc Bonferroni test. * indicates p<0.05, ** indicates p<0.01, *** indicates p<0.001.

FIG. 23 illustrates apoptosis of CS-PGE₂-PINC-treated and CS-PINC-treated NRCMs after being exposed to serum-free IMDM medium supplemented with hydrogen peroxide (250 μM) for 3 h, as determined by TUNEL staining. All data are mean±s.d. (n=3). Comparisons between any two groups were performed using two-tailed unpaired Student's t-test. * indicates p<0.05, ** indicates p<0.01, *** indicates p<0.001.

FIGS. 24A-24C illustrate PINC injection does not stimulate local T-lymphocyte and macrophage immune response. The infiltration of T-lymphocytes with CD3 (FIG. 24A) and CD8 (FIG. 24B) expression was negligible. Macrophages that labeled with CD68 (FIG. 24C) were also barely detected. Nuclei were counterstained with DAPI. Scale bars, 50 μm.

FIG. 25 illustrates representative images showing that cycling cardiomyocytes as indicated by α-SA and Ki67 double-positive staining were barely detected in the peri-infarct region of the hearts treated with saline at week 4. Scale bar, 50 μm.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “ includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

Prior to describing the various embodiments, the following definitions are provided and should be used unless otherwise indicated.

Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.” “About” as used herein indicates that a number, amount, time, etc., is not exact or certain but reasonably close to or almost the same as the stated value. Therefore, the term “about” means plus or minus 0.1 to 50%, 5-50%, or 10-40%, preferably 10-20%, more preferably 10% or 15%, of the number to which reference is being made. Further, it is to be understood that “a”, “an,” and “the” include plural referents unless the content clearly dictates otherwise. Thus, for example, reference to a composition comprising “a compound” includes a mixture of two or more compounds.

### ABBREVIATIONS

Mesenchymal stem cell, MSC; Mesenchymal stem cell/red blood cell membrane-coated inspired Nanoparticle, MRIN, nanoparticle, NP; Mesenchymal Stem Cell/Red Blood Cell (MSC/RBC);

### DEFINITIONS

The terms “administration of” and “administering” a compound or composition as used herein refers to providing a composition of the disclosure to the individual in need of treatment. The composition of the present disclosure may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

The term “allogeneic” refers to deriving from or originating in another subject or patient. An “allogeneic transplant” refers to collection (e.g., isolation) and transplantation of the cells or organs from one subject into the body of another. In some instances, an “allogeneic transplant” includes cells grown or cultured from another subject's cells.

The term “autologous” refers to deriving from or originating in the same subject or patient. An “autologous transplant” refers to collection (e.g., isolation) and re-transplantation of a subject's own cells or organs. In some instances, an “autologous transplant” includes cells grown or cultured from a subject's own cells. For example, in the methods of the present disclosure, the cardiac stem cells may be derived from a cardiac tissue sample excised from the heart of the patient to be treated, cultured, engineered to be fused with platelet membrane vesicles according to the methods of the disclosure and then administered to the same patient for the treatment of a cardiovascular injury therein.

The term “biocompatible” as used herein refers to a material that does not elicit any undesirable local or systemic effects in vivo.

The terms “biological material” and “biological tissue” as used herein refer to cells or tissue in vivo (e.g. cells or tissue of a subject) and in vitro (e.g. cultured cells).

The term “biodegradable” as used herein refers to a polymer or polymer backbone that undergoes with the passage of time substantial degradation under physiological conditions or in a biological environment. In other words, the polymer backbone has a molecular structure that is susceptible to break down (i.e. a reduction in molecular weight) by chemical decomposition in a biological environment (e.g. within a subject or in contact with biological material such as blood, tissue, etc.), as opposed to physical degradation. Such chemical decomposition will typically be via the hydrolysis of labile or biodegradable moieties that form part of the molecular structure of the backbone. Accordingly, such labile or biodegradable moieties will generally be susceptible to hydrolytic cleavage.

Biodegradable and biocompatible polymers have been designated as probable carriers for long term and short time delivery vehicles including non-hydrolysable polymeric conjugates. PEGs and PEOs are the most common hydroxyl end polymers with a wide range of molecular weights to choose for the purpose of solubility (easy carrier mode), degradation times and ease of conjugation. End-protected methoxy-PEGs will also be employed as a straight chain carrier capable of swelling and thereby reducing the chances of getting protein attached or stuck during the subcellular transportation. Certain copolymers of ethylene and vinyl acetate, i.e. EVAc which have exceptionally good biocompatibility, low crystallinity and hydrophobic in nature are candidates. Among the most common and recommended biodegradable polymers from lactic and glycolic acids can be used.

The term “biomimetic” as used herein refers to a material or structure designed to resemble and/or function in a manner similar to a cell found in a native state in an animal or human. In the embodiments of the disclosure, the biomimetic compositions herein disclosed are suitable as substitutes for the replacement of a population of stem cells such as, but not limited to mesenchymal stem cells.

The term “biomolecule species” as used herein refers to any molecule that may be of biological origin and/or interact with a cell in contact therewith. A biomolecule species of use in the microparticles of the disclosure may be, but are not to be limited to, a protein, a polypeptide, a peptide, a nucleic acid molecule, a saccharide, a polysaccharide, a cytokine and the like that may be, but is not limited to, increasing or decreasing the proliferation of the cell or cell line, may sustain viability and/or proliferation of the cell or cell line, or may initiate a change in the cell type from a stem cell type, a precursor cell type or a progenitor cell type.

The terms “cardiac progenitor cells” and “cardiac stem cells” as used herein can refer to a population of progenitor cells derived from human heart tissue. In some aspects the present disclosure, of the cardiac progenitor (stem) cells, at least 3%, 5%, 7%, 10%, 12%, or 15%, e.g., 3-50%, 3-20%, 3-10%, 5-30%, 5-10%, etc., of the cells express Isl1. In some aspects, CPCs comprise about 10%, 15%, 20%, 30%, 40%, or 50%, e.g., 10-50%, 10-40%, 10-30%, 15-40%, etc., GATA4 expressing cells. In some aspects, cardiac stem cells comprise about 5%, 8%, 10%, 13%, or 15%, e.g., 8-15%, 5-15%, 5-13%, etc., NKX2.5 expressing cells. Before fusion with platelet membrane vesicles by the methods of the present disclosure, cardiac stem cells are unmodified cells in that recombinant nucleic acids or proteins have not been introduced into them or the Sca-1⁺, CD45− cell from which it is derived. As such, cardiac stem cells as isolated from cardiac tissue are non-transgenic, or in other words have not been genetically modified. For example, expression of genes such as Isl1, GATA4, and NKX2.5 in CPCs is from the endogenous gene. Cardiac stem cells may comprise Sca-1+, CD45− cells, c-kit⁺ cells, CD90⁺ cells, CD133⁺ cells, CD31⁺ cells, Flk1⁺ cells, GATA4⁺ cells, or NKX2.5+ cells, or combinations thereof. In some aspects, cardiac stem cells comprise about 50% GATA4 expressing cells. In some aspects, cardiac stem cells comprise about 15% NKX2.5 expressing cells. Cardiac stem cells can replicate and are capable of differentiating into endothelial cells, cardiomyocytes, smooth muscle cells, and the like.

The term “cardiac cells” as used herein refers to any cells present in the heart that provide a cardiac function, such as heart contraction or blood supply, or otherwise serve to maintain the structure of the heart. Cardiac cells as used herein encompass cells that exist in the epicardium, myocardium or endocardium of the heart. Cardiac cells also include, for example, cardiac muscle cells or cardiomyocytes; cells of the cardiac vasculatures, such as cells of a coronary artery or vein. Other non-limiting examples of cardiac cells include epithelial cells, endothelial cells, fibroblasts, cardiac conducting cells and cardiac pacemaking cells that constitute the cardiac muscle, blood vessels and cardiac cell supporting structure.

The term “cardiac function” as used herein refers to the function of the heart, including global and regional functions of the heart. The term “global” cardiac function as used herein refers to function of the heart as a whole. Such function can be measured by, for example, stroke volume, ejection fraction, cardiac output, cardiac contractility, etc. The term “regional cardiac function” refers to the function of a portion or region of the heart. Such regional function can be measured, for example, by wall thickening, wall motion, myocardial mass, segmental shortening, ventricular remodeling, new muscle formation, the percentage of cardiac cell proliferation and programmed cell death, angiogenesis and the size of fibrous and infarct tissue. Techniques for assessing global and regional cardiac function are known in the art. For example, techniques that can be used to measure regional and global cardiac function include, but are not limited to, echocardiography (e.g., transthoracic echocardiogram, transesophageal echocardiogram or 3D echocardiography), cardiac angiography and hemodynamics, radionuclide imaging, magnetic resonance imaging (MRI), sonomicrometry and histological techniques.

The term “cardiac tissue” as used herein refers to tissue of the heart, for example, the epicardium, myocardium or endocardium, or portion thereof, of the heart. The term “injured” cardiac tissue as used herein refers to a cardiac tissue that is, for example, ischemic, infarcted, reperfused, or otherwise focally or diffusely injured or diseased. Injuries associated with a cardiac tissue include any areas of abnormal tissue in the heart, including any areas caused by a disease, disorder or injury and includes damage to the epicardium, endocardium and/or myocardium. Non-limiting examples of causes of cardiac tissue injuries include acute or chronic stress (e.g., systemic hypertension, pulmonary hypertension or valve dysfunction), atheromatous disorders of blood vessels (e.g., coronary artery disease), ischemia, infarction, inflammatory disease and cardiomyopathies or myocarditis.

The term “cardiomyocyte cell” refers to a cell comprising striated muscle of the walls of the heart. Cardiomyocytes can contain one or more nuclei.

The term “cardiosphere” refers to a cluster of cells derived from heart tissue or heart cells. In some instances, a cardiosphere includes cells that express stem cell markers (e.g., c-Kit, Sca-1, and the like) and differentiating cells expressing myocyte proteins and the gap protein (connexin 43).

The term “cell” as used herein refers to an animal or human cell. The mesenchymal cells of the disclosure can be removed (isolated) from a tissue and may be cultured to increase the population size of the cells. The animal or human cells may be maintained in a viable state either by culturing by serial passage in a culture medium or cryopreserved by methods well-known in the art. The cells may be obtained from the animal or human individual that has received an injury desired to be repaired by administration of the engineered cells of the disclosure or from a different individual.

Cells, and their extracellular vesicle such as an exosomes that are contemplated for use in the methods of the present disclosure may be derived from the same subject to be treated (autologous to the subject) or they may be derived from a different subject, preferably of the same species, (allogeneic to the subject).

Commercially available media may be used for the growth, culture and maintenance of mesenchymal stem cells. Such media include but are not limited to Dulbecco's modified Eagle's medium (DMEM). Components in such media that are useful for the growth, culture and maintenance of mesenchymal stem cells include but are not limited to amino acids, vitamins, a carbon source (natural and non-natural), salts, sugars, plant derived hydrolysates, sodium pyruvate, surfactants, ammonia, lipids, hormones or growth factors, buffers, non-natural amino acids, sugar precursors, indicators, nucleosides and/or nucleotides, butyrate or organics, DMSO, animal derived products, gene inducers, non-natural sugars, regulators of intracellular pH, betaine or osmoprotectant, trace elements, minerals, non-natural vitamins. Additional components that can be used to supplement a commercially available tissue culture medium include, for example, animal serum (e.g., fetal bovine serum (FBS), fetal calf serum (FCS), horse serum (HS)), antibiotics (e.g., including but not limited to, penicillin, streptomycin, neomycin sulfate, amphotericin B, blasticidin, chloramphenicol, amoxicillin, bacitracin, bleomycin, cephalosporin, chlortetracycline, zeocin, and puromycin), and glutamine (e.g., L-glutamine). Mesenchymal stem cell survival and growth also depends on the maintenance of an appropriate aerobic environment, pH, and temperature. Mesenchymal stem cells can be maintained using methods known in the art (see, for example, Pittenger et al., (1999) Science 284:143-147).

The term “cell therapy” as used herein refers to the introduction of new cells into a tissue in order to treat a disease and represents a method for repairing or replacing diseased tissue with healthy tissue.

The term “clonal” refers to a cell or a group of cells that have arisen from a single cell through numerous cycles of cell division. The cells of a clonal population are genetically identical. A clonal population can be a heterogeneous population such that the cells can express a different set of genes at a specific point in time.

The term “culturing” as used herein refers to growing cells or tissue under controlled conditions suitable for survival, generally outside the body (e.g., ex vivo or in vitro). The term includes “expanding,” “passaging,” “maintaining,” etc. when referring to cell culture of the process of culturing. Culturing cells can result in cell growth, differentiation, and/or division.

The term “derived from” as used herein refers to cells or a biological sample (e.g., blood, tissue, bodily fluids, etc.) and indicates that the cells or the biological sample were obtained from the stated source at some point in time. For example, a cell derived from an individual can represent a primary cell obtained directly from the individual (i.e., unmodified). In some instances, a cell derived from a given source undergoes one or more rounds of cell division and/or cell differentiation such that the original cell no longer exists, but the continuing cell (e.g., daughter cells from all generations) will be understood to be derived from the same source. The term includes directly obtained from, isolated and cultured, or obtained, frozen, and thawed. The term “derived from” may also refer to a component or fragment of a cell obtained from a tissue or cell, including, but not limited to, a protein, a nucleic acid, a membrane or fragment of a membrane, and the like.

The term “disaggregating” includes separating, dislodging, or dissociating cells or tissue using mechanical or enzymatic disruption to isolate single cells or small clusters of cells. In some instances, enzymatic disruption can be replaced with one of more enzyme alternatives having substantially the same effect as the enzyme.

The term “endothelial cell” refers to a cell necessary for the formation and development of new blood vessel from pre-existing vessels (e.g., angiogenesis). Typically, endothelial cells are the thin layer of cells that line the interior surface of blood vessels and lymphatic vessels. Endothelial cells are involved in various aspects of vascular biology, including atherosclerosis, blood clotting, inflammation, angiogenesis, and control of blood pressure.

The term “extracellular vesicle” as used herein can refers to a membrane vesicle secreted by cells that may have a larger diameter than that referred to as an “exosome”. Extracellular vesicles (alternatively named “microvesicle” or “membrane vesicle”) may have a diameter (or largest dimension where the particle is not spheroid) of between about 10 nm to about 5000 nm (e.g., between about 50 nm and 1500 nm, between about 75 nm and 1500 nm, between about 75 nm and 1250 nm, between about 50 nm and 1250 nm, between about 30 nm and 1000 nm, between about 50 nm and 1000 nm, between about 100 nm and 1000 nm, between about 50 nm and 750 nm, etc.). Typically, at least part of the membrane of the extracellular vesicle is directly obtained from a cell (also known as a donor cell). Extracellular vesicles suitable for use in the compositions and methods of the present disclosure may originate from cells by membrane inversion, exocytosis, shedding, blebbing, and/or budding. Extracellular vesicles may originate from the same population of donor cells yet different subpopulations of extracellular vesicles may exhibit different surface/lipid characteristics. Alternative names for extracellular vesicles include, but are not limited to, exosomes, ectosomes, membrane particles, exosome-like particles, and apoptotic vesicles. Depending on the manner of generation (e.g., membrane inversion, exocytosis, shedding, or budding), the extracellular vesicles contemplated herein may exhibit different surface/lipid characteristics.

The term “exosomes” as used herein refers to small secreted vesicles (typically about 30 nm to about 150 nm (or largest dimension where the particle is not spheroid)) that may contain, or have present in their membrane, nucleic acid, protein, or other biomolecules and may serve as carriers of this cargo between diverse locations in a body or biological system. The term “exosomes” as used herein advantageously refers to extracellular vesicles that can have therapeutic properties, including, but not limited to stem cell exosomes such as cardiac stem cell or mesenchymal stem cells.

Exosomes may be isolated from a variety of biological sources including mammals such as mice, rats, guinea pigs, rabbits, dogs, cats, bovine, horses, goats, sheep, primates or humans. Exosomes can be isolated from biological fluids such as serum, plasma, whole blood, urine, saliva, breast milk, tears, sweat, joint fluid, cerebrospinal fluid, semen, vaginal fluid, ascetic fluid and amniotic fluid. Exosomes may also be isolated from experimental samples such as media taken from cultured cells (“conditioned media”, cell media, and cell culture media).

Exosomes may also be isolated from tissue samples such as surgical samples, biopsy samples, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes.

Exosomes may be isolated from freshly collected samples or from samples that have been stored frozen or refrigerated. Although not necessary, higher purity exosomes may be obtained if fluid samples are clarified before precipitation with a volume-excluding polymer, to remove any debris from the sample. Methods of clarification include centrifugation, ultracentrifugation, filtration or ultrafiltration.

The genetic information within the extracellular vesicle such as an exosome may easily be transmitted by fusing to the membranes of recipient cells, and releasing the genetic information into the cell intracellularly. Though exosomes as a general class of compounds represent great therapeutic potential, the general population of exosomes are a combination of several class of nucleic acids and proteins which have a constellation of biologic effects both advantageous and deleterious. In fact, there are over 1000 different types of exosomes.

The terms “generate”, “generation”, and “generating” as used herein shall be given their ordinary meaning and shall refer to the production of new cells in a subject and optionally the further differentiation into mature, functioning cells. Generation of cells may comprise regeneration of the cells. Generation of cells comprises improving survival, engraftment and/or proliferation of the cells.

The term “growth factors” as used herein refers to proteins, peptides or other molecules having a growth, proliferative, differentiation, or trophic effect on cells. Such factors may be used for inducing proliferation or differentiation and can include, for example, any trophic factor that allows cells to proliferate, including any molecule which binds to a receptor on the surface of the cell to exert a trophic, or growth-inducing effect on the cell. Such factors include paracrine factors secreted by stem cells and which may induce or sustain proliferation or differentiation by cells in close proximity to the biomimetic microparticles of the disclosure. Factors include, but are not limited to, Adrenomedullin (AM); Angiopoietin (Ang); Autocrine motility factor; Bone morphogenetic proteins (BMPs); Ciliary neurotrophic factor family; Ciliary neurotrophic factor (CNTF); Leukemia inhibitory factor (LIF); Interleukin-6 (IL-6); Colony-stimulating factors; Macrophage colony-stimulating factor (m-CSF); Granulocyte colony-stimulating factor (G-CSF); Granulocyte macrophage colony-stimulating factor (GM-CSF); Epidermal growth factor (EGF); Ephrin A1; Ephrin A2; Ephrin A3; Ephrin A4; Ephrin A5; Ephrin B1; Ephrin B2; Ephrin B3; Erythropoietin (EPO); Fibroblast growth factor (FGF); Foetal Bovine Somatotrophin (FBS); GDNF family of ligands: Glial cell line-derived neurotrophic factor (GDNF); Neurturin; Persephin; Artemin; Growth differentiation factor-9 (GDF9); Hepatocyte growth factor (HGF); Hepatoma-derived growth factor (HDGF); Insulin; Insulin-like growth factor-1 (IGF-1); Insulin-like growth factor-2 (IGF-2); Interleukins: IL-1; IL-2; IL-3; IL-4; IL-5; IL-6; IL-7; Keratinocyte growth factor (KGF); Migration-stimulating factor (MSF); Macrophage-stimulating protein (MSP); Myostatin (GDF-8); Neuregulins; Neuregulin 1 (NRG1); Neuregulin 2 (NRG2); Neuregulin 3 (NRG3); Neuregulin 4 (NRG4); Brain-derived neurotrophic factor (BDNF); Nerve growth factor (NGF); Neurotrophin-3 (NT-3); Neurotrophin-4 (NT-4); Placental growth factor (PGF); Platelet-derived growth factor (PDGF); Renalase (RNLS); T-cell growth factor (TCGF); Thrombopoietin (TPO); Transforming growth factors; Transforming growth factor alpha (TGF-α); Transforming growth factor beta (TGF-β); Tumor necrosis factor-alpha (TNF-α); Vascular endothelial growth factor (VEGF), and the like.

The term “isolating” or “isolated” when referring to a cell or a molecule (e.g., nucleic acids or protein) indicates that the cell or molecule is or has been separated from its natural, original or previous environment. For example, an isolated cell can be removed from a tissue derived from its host individual, but can exist in the presence of other cells (e.g., in culture), or be reintroduced into its host individual.

The term “mesenchymal stem cells (MSCs)” as used herein refers to progenitor cells having the capacity to differentiate into neuronal cells, adipocytes, chondrocytes, osteoblasts, myocytes, cardiac tissue, and other endothelial and epithelial cells. (See for example Wang, (2004) Stem Cells 22: 1330-1337; McElreavey (1991) Biochem. Soc. Trans. 1: 29s; Takechi (1993) Placenta 14: 235-245; Yen (2005) Stem Cells; 23: 3-9). These cells have been characterized to express, and thus be positive for, one or more of CD13, CD29, CD44, CD49a, b, c, e, f, CD51, CD54, CD58, CD71, CD73, CD90, CD102, CD105, CD106, CDw119, CD120a, CD120b, CD123, CD124, CD126, CD127, CD140a, CD166, P75, TGF-βIR, TGF-βIIR, HLA-A, B, C, SSEA-3, SSEA-4, D7 and PD-L1.

Mesenchymal stem cells may be harvested from a number of sources including, but not limited to, bone marrow, blood, periosteum, dermis, umbilical cord blood and/or matrix (e.g., Wharton's Jelly), and placenta. Example of methods for obtaining mesenchymal stem cells reference can be found, for example, in U.S. Pat. No. 5,486,359.

The terms “modulating the proliferative status” or “modulating the proliferation” as used herein refers to the ability of a compound to alter the proliferation rate of a population of stem or progenitor cells. A compound may be toxic, wherein the proliferation of the cells is slowed or halted, or the proliferation may be enhanced such as, for example, by the addition to the cells of a cytokine or growth factor.

The term “multipotent” refers to a cell having the potential to differentiate into multiple, yet a limited number of cell types or cell lineages. Typically, these cells are considered unspecialized cells that have the ability to self-renew and become specialized cells with specific functions and characteristics.

The term “paracrine signaling” as used herein refers to a form of cell-cell communication in which a cell produces a signal to induce changes in nearby cells, altering the behavior or differentiation of those cells. Signaling molecules known as paracrine factors diffuse over a relatively short distance (local action), as opposed to endocrine factors (hormones which travel considerably longer distances via the circulatory system), juxtacrine interactions, and autocrine signaling. Cells that produce paracrine factors secrete them into the immediate extracellular environment. Factors then travel to nearby cells in which the gradient of factor received determines the outcome

Although paracrine signaling elicits a diverse array of responses in the induced cells, most paracrine factors utilize a relatively narrow set of receptors and pathways. Different organs in the body, even between different species, can utilize similar sets of paracrine factors in differential development. Highly conserved receptors and pathways can be organized into four major families based on similar structures: Fibroblast growth factor (FGF) family, Hedgehog family, Wnt family, and TGF-β superfamily. Binding of a paracrine factor to its respective receptor initiates signal transduction cascades, eliciting different responses.

For paracrine factors to induce a response in the receiving cell, that cell must have the appropriate receptors available on the cell membrane to receive the signals, also known as being competent. Additionally, the responding cell must also have the ability to be mechanistically induced.

The term “PGE2” as used herein refers to prostaglandin E2 (5Z,11a,13E,15S)-11,15-Dihydroxy-9-oxo-prosta-5,13-dien-1-oic acid), a potent activator of the Wnt signaling pathway. It has been implicated in regulating the developmental specification and regeneration of hematopoietic stem cells through cAMP/PKA activity.

The term “pharmaceutically acceptable carrier, excipient, or vehicle” as used herein refers to a medium which does not interfere with the effectiveness or activity of an active ingredient and which is not toxic to the hosts to which it is administered and which is approved by a regulatory agency of the Federal or a state government or listed in the U.S. Pharmacopeia or other generally recognized pharmacopeia for use in animals, and more particularly in humans. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

The terms “pharmaceutically acceptable” or “pharmacologically acceptable” as used herein refer to a material that is not biologically or otherwise undesirable, i.e., the material may be administered to an individual in a formulation or composition without causing any undesirable biological effects or interacting in a deleterious manner with any of the components of the composition in which it is contained.

The term “polymer” as used herein refers to molecules comprising two or more monomer subunits that may be identical repeating subunits or different repeating subunits. A monomer generally comprises a simple structure, low-molecular weight molecule containing carbon. Polymers may optionally be substituted. Polymers that can be used in the present disclosure include without limitation vinyl, acryl, styrene, carbohydrate derived polymers, polyethylene glycol (PEG), polyoxyethylene, polymethylene glycol, poly-trimethylene glycols, polyvinylpyrrolidone, polyoxyethylene, polyoxypropylene block polymers, and copolymers, salts, and derivatives thereof. In aspects of the disclosure, the polymer is poly(2-acrylamido-2-methyl-1-propanesulfonic acid); poly(2-acrylamido-2-methyl,-1-propanesulfonic acid-coacrylonitrile, poly(2-acrylamido-2-methyl,-1-propanesulfonic acid-co-styrene), poly(vinylsulfonic acid); poly(sodium 4-styrenesulfonic acid); and sulfates and sulfonates derived therefrom; poly(acrylic acid), poly(methylacrylate), poly(methyl methacrylate), and polyvinyl alcohol).

The term “progenitor cell” as used herein refers to a cell that has the capacity to differentiate into a specific type of cell, as well as replicate to generate a daughter cell substantially equivalent to itself. In some instances, a progenitor cell undergoes limited self-renewal such that it does not self-replicate indefinitely.

The term “proliferative status” as used herein refers to whether a population of cells including, but not limited to, mesenchymal stem or progenitor cells, or a subpopulation thereof, are dividing and thereby increasing in number, in the quiescent state, or whether the cells are not proliferating, dying or undergoing apoptosis.

The terms “regenerate,” “regeneration” and “regenerating” as used herein refer to the process of growing and/or developing new tissue to replace tissue that has been injured, for example, by viral infection, chemically-induced injury, or other disease. Tissue regeneration may comprise activation and/or enhancement of cell proliferation.

The term “secretome” as used herein refers to polypeptides secreted into and collected from an extracellular culture medium and include growth factors, chemokines, cytokines, adhesion molecules, proteases and shed receptors. Human protein-coding genes (39%, 19613 genes) are predicted to have either a signal peptide and/or at least one transmembrane region suggesting active transport of the corresponding protein out of the cell (secretion) or location in one of the numerous membrane systems in the cell. Non-protein components, such as lipid, micro-RNAs and messenger-RNA can also be secreted by cells via both microvesicles (100 to more than 1000 nm diameter) shed from the plasma membrane and exosomes (from about 30 nm to about 150 nm diameter) that are released via endosomal-exocytosis event. Factors present in both these organelles accounts for up to 42% of the secretome and are included in the collective secretome. Accordingly, the conditioned cell culture media of the present disclosure may include some or all secreted proteins, nucleic acids, microvesicles, and exosomes generated from a population of cells, including but not limited to, or isolated examples of such.

The term “self-renewal” or “self-renewing” as used herein refers to the ability of a cell to divide through numerous cycles of cell division and generate a daughter with the same characteristics as the parent cell. The other daughter cell can have characteristics different from its parent cell. The term includes the ability of a cell to generate an identical genetic copy of itself (e.g., clone) by cell division. For example, a self-renewing cardiac progenitor cell can divide to form one daughter cardiac progenitor cell and another daughter cell committed to differentiation to a cardiac lineage such as an endothelial, smooth muscle or cardiomyocyte cell. In some instances, a self-renewing cell does not undergo cell division forever.

The term “smooth muscle cell” refers to a cell comprising non-striated muscle (e.g., smooth muscle). Smooth muscle is present within the walls of blood vessels, lymphatic vessels, cardiac muscle, urinary bladder, uterus, reproductive tracts, gastrointestinal tract, respiratory tract, and iris of the eye.

The term “stem cell” as used herein refers to cells that have the capacity to self-renew and to generate differentiated progeny. The term “pluripotent stem cells” refers to stem cells that have complete differentiation versatility, i.e., the capacity to grow into any of the fetal or adult mammalian body's approximately 260 cell types. For example, pluripotent stem cells have the potential to differentiate into three germ layers: endoderm (e.g., blood vessels), mesoderm (e.g., muscle, bone and blood) and ectoderm (e.g., epidermal tissues and nervous system), and therefore, can give rise to any fetal or adult cell type.

The terms “subject,” “patient,” “individual” and the like are used interchangeably and refer to, except where indicated, mammals such as humans and non-human primates, as well as rabbits, rats, mice, dogs, cats, goats, pigs, cows, and other mammalian species. The term does not necessarily indicate that the subject has been diagnosed with a particular disease, but typically refers to an individual under medical supervision.

The term “tissue injury” as used herein refers to damage to a vascularized tissue of an animal or human, wherein the damage is adjacent to, or in close proximity to, a blood vessel that has also undergone injury, and in particular loss of endothelial cells lining the lumen of the blood vessel. For example, but not intended to be limiting, vascular ischemia can result in both loss of vascular endothelial cells to expose the underlying subendothelial matrix. The loss of adequate blood flow can result in loss of cell viability in such as cardiac tissue, brain or neurological tissue that is in contact with the occluded blood vessel.

The term “transplant” as used herein refers to cells, e.g., cardiac progenitor cells, introduced into a subject. The source of the transplanted material can include cultured cells, cells from another individual, or cells from the same individual (e.g., after the cells are cultured, enriched, or expanded ex vivo or in vitro).

The terms “treatment,” “therapy,” “amelioration” and the like refer to any reduction in the severity of symptoms. As used herein, the terms “treat” and “prevent” are not intended to be absolute terms. Treatment can refer to any delay in onset, amelioration of symptoms, improvement in patient survival, repair/regeneration of heart tissue or blood vessels, increase in survival time or rate, etc. The effect of treatment can be compared to an individual or pool of individuals not receiving the treatment. In some instances, the effect can be the same patient prior to treatment or at a different time during the course of therapy. In some aspects, the severity of disease, disorder or injury is reduced by at least 10%, as compared, e.g., to the individual before administration or to a control individual (e.g., healthy individual or an individual no longer having the disease, disorder or injury) not undergoing treatment. In some instances, the severity of disease, disorder or injury is reduced by at least 20%, 25%, 50%, 75%, 80%, or 90%. In some cases, the symptoms or severity of disease are no longer detectable using standard diagnostic techniques.

### Description

The present disclosure encompasses embodiments of a mesenchymal stem cell (MSC)/red blood cell (RBC) membrane-coated inspired nanoparticle (or MRIN). Thus, a MRIN has a MSC-derived core and a RBC membrane shell. The MSC core can comprise the therapeutic MSC secretome, encapsulated in a poly (lactic-co-glycolic acid) (PLGA). To increase stability, this particle is further encased in RBC membranes to make the final MRIN product. In the present disclosure, MRINs were fabricated and their stability, biodistribution, and therapeutic potency tested in a murine model of acute liver failure.

A nanoparticle of the disclosure, termed a MRIN, was fabricated by coating RBC cell membranes onto PLGA particles loaded with MSC secretome. This nanoparticle exhibited both a secretome comparable to that of intact viable MSCs and a surface antigen profiles of RBCs. The advantages of the system of the disclosure are: 1) the RBC membrane serves as the camouflage to protect the nanoparticle from recognition by macrophages; 2) the size of the MRIN, about 200 nm, can be tailored to have great liver retention after intravenous delivery; 3) unlike intact MSCs, MRINs to tolerate long-term cryo-storage after lyophilization.

Previous work reported micro-sized synthetic MSC particles (MSC secretome+MSC membrane) (Luo et al., (2017) Circ. Res. 120: 1768-1775). However, the MRINs of the disclosure have increased stability and bioavailability to the liver after intravenous delivery since 1) micro-sized particles will be blocked by the lungs after intravenous delivery. Therefore, the MRINs of the disclosure are designed as a nanoparticle; and 2) since the MRINs of the disclosure will circulate in the blood, a (MSC secretome+RBC membrane) strategy was adopted for improved hemocompatibility.

The MRINs of the disclosure integrate stem cell biology and nanomedicine. Cell therapy is a promising approach to liver regeneration. So far, multiple cell types including MSCs, have been tested preclinically and clinically for treating liver diseases such as liver failure, liver fibrosis or liver cirrhosis (Lin et al., (2017) Hepatology 66: 209-2019; Zhang et al., (2014) Hepatology 59: 671-682; El Agha et al., (2017) Cell Stem Cell 21: 166-177). However, translation and efficacy are hindered by the intrinsic limitations of cellular products. Intravenously-injected cells are filtered in the lungs and diminished capacity to reach the liver. In addition, cell viability can rapidly decrease over the time during storage and shipping. These dead or dying cells can produce pro-death microvesicles that can undermine the overall therapeutic benefits of the cell therapy.

To overcome these acknowledged deficiencies in intact cell therapy, the biomimetic approach of the disclosure was taken by generating polymer nanoparticles (NPs) that encapsulate the growth factors secreted by MSCs. Unlike intact viable MSCs, these particles (approximately 200 nm in diameter) are small enough to pass the lungs and reach the liver. In addition, since they are not intact viable cells, they can be lyophilized and cryo-stored before usage. To make the NP more stable in the blood and in the liver, the NPs were cloaked with the membranes derived from RBCs. The final product MRIN integrates the advantages of MSCs, RBCs, and nanoparticles.

A “core-shell” therapeutic microparticle, therefore, is provided that mimics stem cell biointerfacing during regeneration. This particle, a cell-mimicking microparticle (CMMP), contains control-released stem cell factors in its polymeric core and is cloaked with red blood cell membrane fragments on the surface. While not wishing to be bound to any one theory, it is contemplated that CMMP can exert similar regenerative outcomes as real mesenchymal stem cells but are advantageous over the later since they are more stable during storage and do not stimulate T cell immune reaction since they are not real cells.

Accordingly, provided are embodiments of a polymer microparticle that emulates, for example, mesenchymal stem cell functions during tissue repair. In a mouse model of liver malfunction, injection of CMMPs of the disclosure led to the preservation of viable liver tissue and augmentation of liver functions similar to mesenchymal stem cell therapy. CMMPs (derived from human cells) and coated in red blood cell membranes did not stimulate T cell infiltration in immuno-competent mice, suggesting an advantageous safety profile. Although one application is targeted to the liver, the CMMPs of the disclosure represent a platform technology that can be applied to multiple stem cell types and the repair of a variety of organ systems.

The present disclosure, therefore, encompasses embodiments of a synthetic cell-mimicking microparticle (CMMP) that is useful for recapitulating (mimicking) mesenchymal stem cell functions in tissue repair. The CMMPs of the disclosure can accommodate and deliver secreted proteins and exosomes derived from mesenchymal stem cells. It has been shown, for example, that in a mouse model of chemically-induced liver degeneration, injection of CMMPs of the disclosure will lead to the preservation of viable hepatocytes and augmentation of cardiac functions similar to stem cell therapy. CMMPs (derived from human cells) do not stimulate T cells infiltration in immuno-competent mice. However, the microparticles of the disclosure may be used to mimic a broad range of stem cells that may be usefully applied as therapeutic agents. Accordingly, CMMPs of the disclosure can function as “synthetic stem cells” that mimic the paracrine and biointerfacing activities of natural stem cells in therapeutic cardiac regeneration.

CMMP represents a synthetic microparticle functionalized with mesenchymal stem cell-derived secretome products or fractions thereof. Advantageously, CMMP overcomes several limitations of live stem cells as therapeutic agents. First, living stem cells need to be carefully cryo-preserved and thawed before they can be sent to the clinic. As living organisms, how the cells are prepared and processed can greatly affect the therapeutic outcomes. Second, stem cell transplantation carries certain risks (e.g. tumorigenecity and immunogenicity if allogeneic or xenogeneic cells were used).

It is contemplated that CMMPs of the disclosure can be delivered intravenously, although the CMMPs may be directly delivered to a site of injury and thereby promote extravasation through the mechanism of angiopellosis. In addition, CMMP represents a technology that can be widely applied to other stem cell types and the repair of various other organ systems.

The present disclosure, therefore, encompasses embodiments of stem cell biomimetic microparticles, including but not limited to mesenchymal stem cell-derived microparticles that comprise at least one mesenchymal stem cell-derived paracrine polypeptide, growth factor, or population of exosomes embedded in a polymer core particle which further comprises an outer layer of at least one fragment of a red blood cell membrane disposed on the core particle. The polymer core itself is constituted of any biocompatible and biodegradable polymer or copolymer, or a combination of polymer or copolymer species that will allow the embedding of the paracrine factors and their prolonged release from the core. The core and hence the microparticles are preferably biodegradable which will lead to their being eventually eliminated from a recipient animal or human subject.

The core particles are sized to allow both transport through blood vessels and extravasation from the blood vessels into the surrounding tissues. The polymer of the core particle may be formed from a solution of the component monomers that further includes culture medium conditioned by mesenchymal stem cell growth therein, at least one polypeptide or peptide growth factor selected to induce the generation and proliferation of a population of mesenchymal stem cells, or a population of mesenchymal stem cell-derived exosomes. Most preferably, the growth factor(s) has been selected as also being secreted by a targeted stem cell population. Accordingly, one source of the polypeptides for incorporation in the biomimetic microparticles of the disclosure is a conditioned culture medium that has been used to culture and increase a population of isolated stem cells.

During the culturing of the cells they are known to generate and secrete paracrine growth factors that can, for example, interact with receptors on the surface of similar stem cells to promote the growth of the recipient cells. Thus, by admixing the cultured medium with the solution of the polymer precursors and then forming a polymer, the growth factors become embedded in the matrix of the polymer of the microparticles. Before admixing with the polymer precursor, the growth factor polypeptides of the conditioned medium can be concentrated to a degree that when embedded in the microparticles and delivered to a recipient animal or human subject they will be secreted from the microparticles to provide a concentration effective in stimulating the growth and proliferation of cells in the target tissue. The constituents of the conditioned medium may be concentrated by methods well known to those of skill in the art, for example, but not intended to be limiting, by such as lyophilization and resuspension in a reduced liquid volume and by ultrafiltration.

Other than conditioned cell culture medium, the microparticles of the disclosure may have embedded therein defined growth factor polypeptides that have been identified as capable of inducing and sustaining stem cell proliferation in vivo. Any combination or number of different such growth factors may be incorporated into the microparticles, the combination being adjusted for the type of stem cell and tissue repair of interest. Accordingly, the conditioned medium as a source of the growth factors may be replaced by a physiologically acceptable solution of isolated growth factors such as those that are readily commercially available

The microparticles of the disclosure may have embedded therein a population of exosomes secreted by the cultured mesenchymal stem cells. Any combination or number of different such growth factors, exosomes, and the like may be incorporated into the microparticles, the combination being adjusted for the type of stem cell and tissue repair of interest. Accordingly, the conditioned medium as a source of the growth factors may be replaced by a physiologically acceptable solution of isolated growth factors such as those that are readily commercially available. Compositions of the present disclosure can include those that comprise a sustained release or controlled release matrix comprising at least one polymer material within which are embedded stem cell-derived paracrine factors. Embodiments of the present disclosure can be used in conjunction with other treatments that use sustained-release formulations. As used herein, a sustained-release microparticle polymer is preferably degradable by enzymatic or acid-based hydrolysis or by dissolution. Once inserted into the recipient tissue, the microparticles can be acted upon by enzymes and body fluids. Polymer matrix desirably is chosen from biocompatible materials such as polylactides (polylactic acid), polyglycolide (polymer of glycolic acid), polylactide co-glycolide (copolymers of lactic acid and glycolic acid), polyanhydrides, poly(ortho)esters, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

The biomimetic microparticles of the disclosure further comprise an outer layer disposed on the outer surface of the microparticles, the layer comprising fragments of the cell membrane derived from red blood cells. While it is preferable that the membrane fragments are isolated from the same species of animal as the type of stem cell used to provide the paracrine factors embedded in the microparticles, it is considered possible to provide a combination where the paracrine factors, exosomes, and the like, are sourced from one species of animal and the membranes from the red blood cells of a different species.

Advantageously, the biomimetic microparticles of the disclosure, prior to the addition of the outer cell membrane layer may be prepared for prolonged storage using such techniques that maintain the stability of embedded growth factors and the integrity of the polymer matrix forming the body of the microparticles. Such technique are well-known to those in the art and include, but are not limited to, lyophilization (freeze-drying), freezing, or in buffered solution, and the like. The preserved microparticles may then, before administering to a recipient animal or human patient, be resuspended in a physiologically acceptable medium and then coated with the fragments of the isolated cell membranes.

A microparticle of the disclosure may be fabricated, for example, by coating red blood cell membranes onto PLGA particles loaded with a MSC secretome. This novel particle can exhibit similar secretome and surface antigen profiles when compared with real MSCs. Such microparticles have been shown to promote hepatic function and display cryopreservation and lyophilization stability in vitro.

Emerging lines of evidence indicate that adult stem cells exert their therapeutic effects mainly through paracrine effects rather than direct differentiation. To that end, the direct delivery of stem cell-released soluble factors has been considered as an alternative approach to stem cell transplantation. However, progress is hindered by the short-lived effect of injected soluble factors. The cardiac contraction can quickly wash away the injected factors. Approaches that allow controlled release of soluble factors are paramount and urgently needed for the clinical implementation of stem cell-derived factors for therapeutic heart regeneration. Although exosomes show advantages for cardiac repair and can overcome the shortcomings associated with cell transplantation, the lack of standardized protocol for exosome isolation and the quick washout of exosomes after injection remains challenging for clinical application.

Accordingly, synMSCs were designed that combined the secretome (containing both soluble factors and exosomes) and membranes of MSC. synMSC can release soluble factors such as vascular endothelial growth factor, stromal cell-derived factor-1, and insulin-like growth factor 1, binding to cardiomyocytes in vitro. In addition, the expression of MHC class I molecules, but not of MHC class II molecules or co-stimulatory molecules, in MSC cell membranes allows it to escape allorecognition by the immune system and may modulate the host immune response. The red blood cell-derived membrane coating on PLGA particles could effectively protect the microparticles from being attacked by host immune and inflammatory cells.

Carbon tetrachloride (CCl₄)-induced acute liver failure is a well-established model to study liver failure in humans (Nagamoto et al., (2016) J. Hepatol. 64: 1068-1075). Accordingly, the therapeutic effects of NP (with MSC factors but without RBC coating), MRIN, and bare conditioned media from MSC were tested in the same model. A PBS injection group was included as a negative control. The highest degree of therapeutic effects was seen in the MRIN-injected animals. While not wishing to be bound by any one theory, the higher retention of MRIN than NP in the liver may not due to active targeting but the result of a longer blood half-life. MRIN has longer circulation time than NP. This resulted in a higher retention in the liver at 12 hrs and 24 hrs. While NPs or bare conditioned media from MSCs could also promote liver regeneration, but to a lesser degree, the regenerative effects were amplified by MRIN due to its excellent stability and longer blood half-life.

It was found that intravenous injections of MRINs (i.e. a nanoparticle composed of MSC-secreted factors and RBC membranes) reduced the circulating levels of pro-inflammatory cytokines, decreased hepatic apoptosis, augmented liver regeneration and function, and eventually improved the survival rates of the mice in acute liver failure.

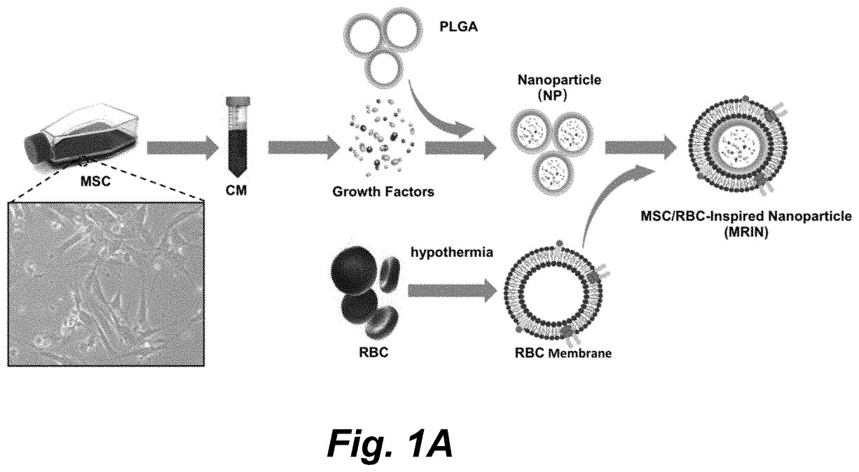

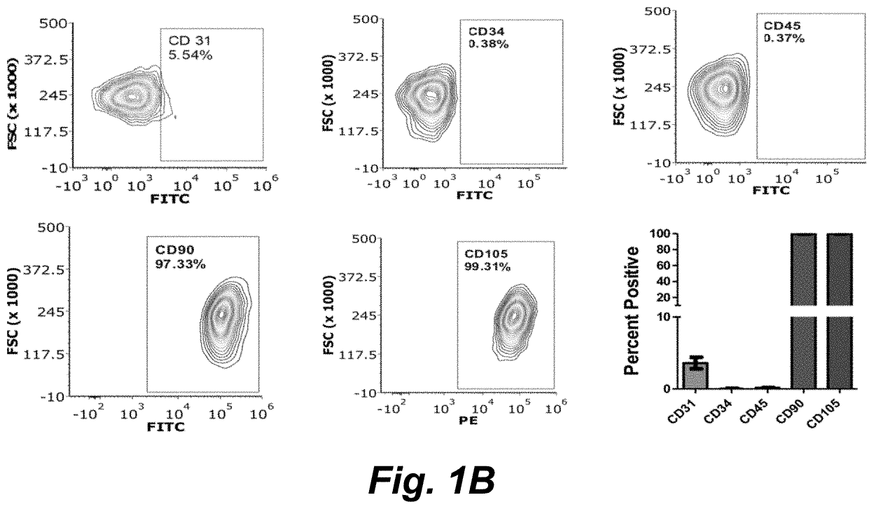

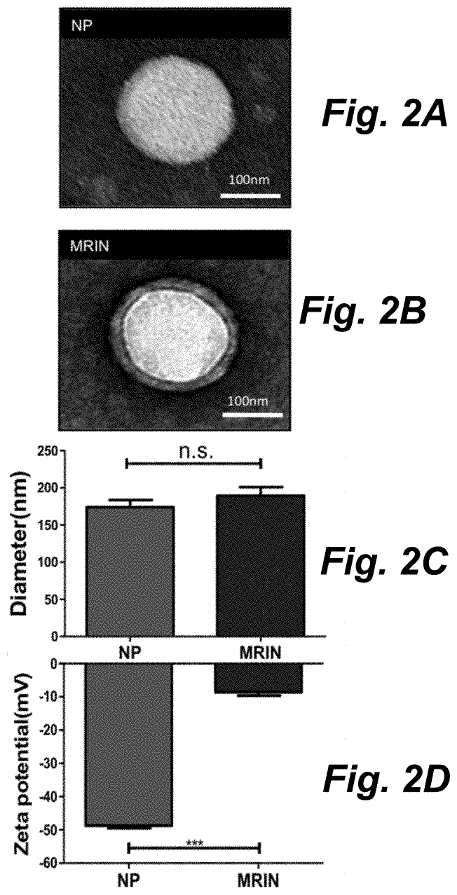

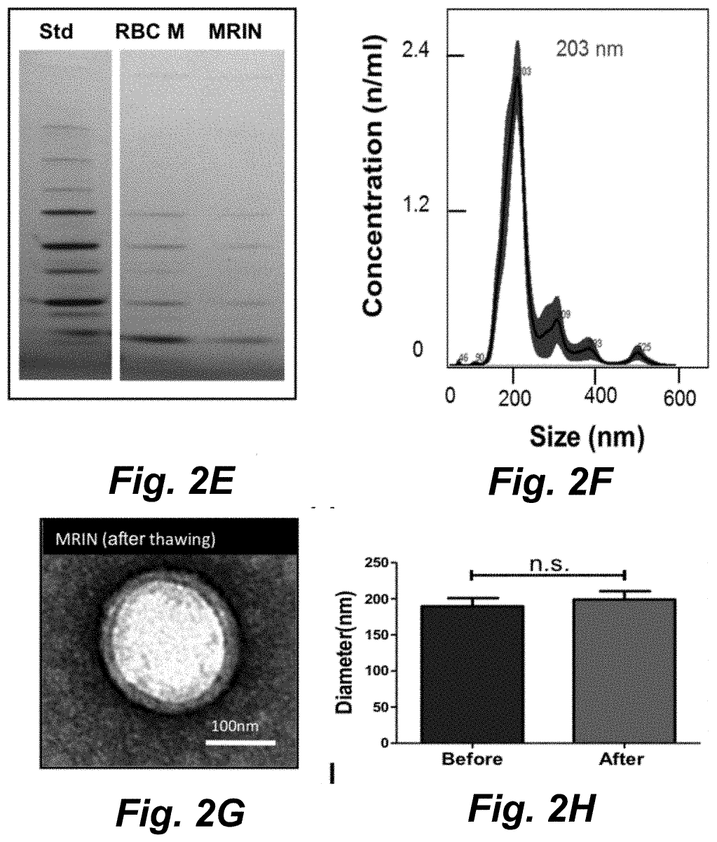

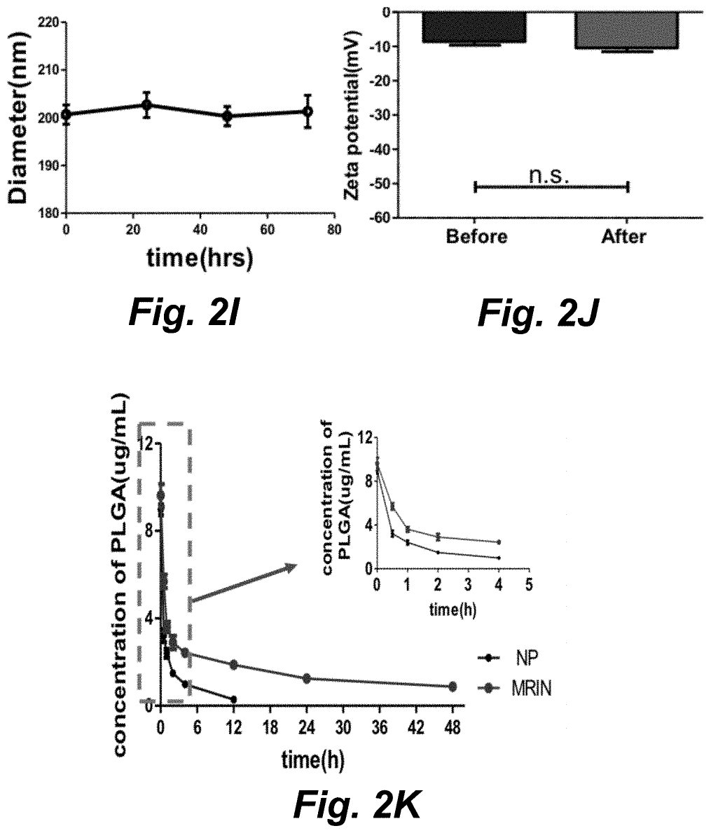

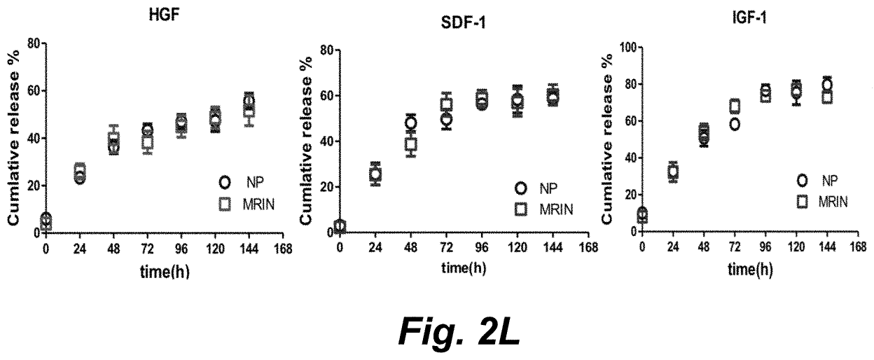

The schematic design of a MRIN according to the disclosure is shown in FIG. 1A. The identity of MSCs was characterized by flow cytometry targeting common MSC markers (FIG. 1B). MSC-conditioned media (CM) was incorporated into PLGA to first form nanoparticles (NPs or MSC-NPs). The NPs could then be further coated with RBC cell membrane vesicles to form the final MSC/RBC-inspired nanoparticle (MRIN). Transmission electron microscopy (TEM) imaging confirmed the RBC membrane coat on MRINs that was not present on NPs (FIGS. 2A and 2B).

NanoSight characterization showed that the size of MRINs generated according to the disclosure was approximately 200 nm (FIGS. 2C and 2F). After RBC membrane cloaking, the size of nanoparticle did not change significantly (FIG. 2C) while the zeta potential of MRIN changed from −47 to −10 mV (FIG. 2D), in accordance with previously reported RBC nanoparticles (Hu et al., (2011) Proc. Natl. Acad. Sci. U.S.A. 108: 10980-10985). This zeta potential is advantageous for intravenous applications. Cryo-preservation did not alter the morphology, size, or zeta potential of the MRINs of the disclosure (FIGS. 2G, 2H, and 2J). The MRINs were stable after long-term storage at room temperature, without significant co-aggregation (FIG. 2I).

SDS-PAGE showed similar protein compositions of RBC membranes and before and after coating the MRINs (FIG. 2E). Intravenously-injected MIRN have a longer blood retention than do NP in mice with acute liver failure (FIG. 2K).

The release profiles of growth factors such as insulin-like growth factor-1 (IGF), stromal cell-derived factor-1 (SDF-1), and hepatocyte growth factor (HGF) (FIG. 2L) were indistinguishable between MRINs and control NPs. This indicates the RBC coating did not affect growth factor releases from MRINs.

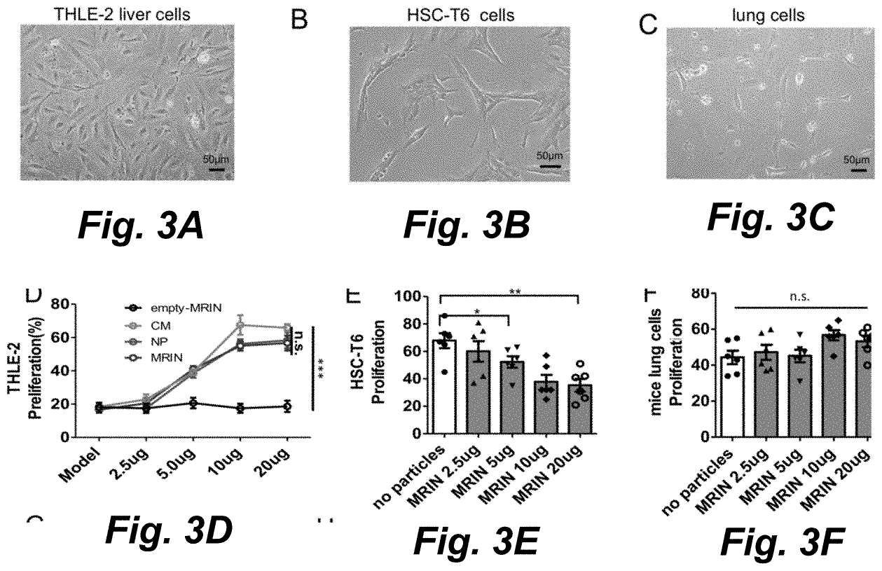

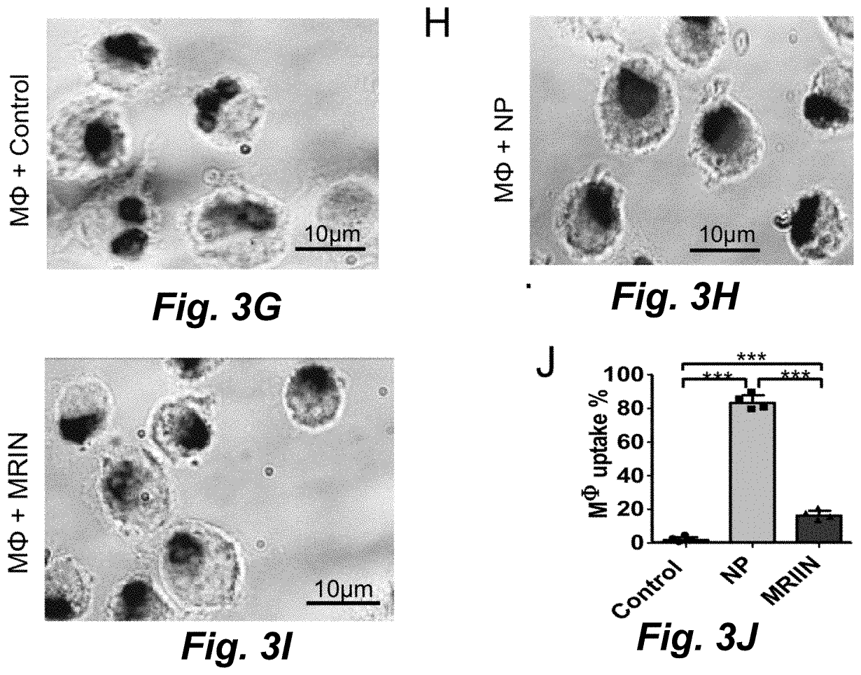

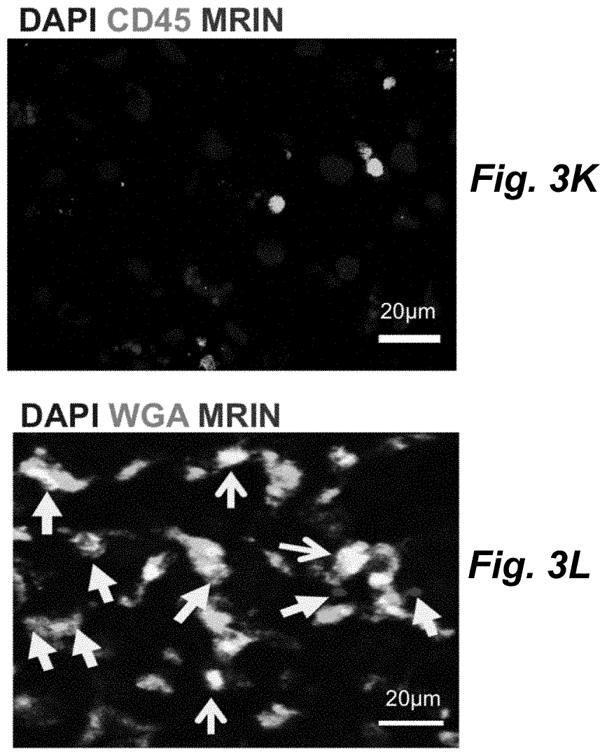

MRINs promote liver cell proliferation while discouraging macrophage uptake: The effects of co-culturing MRIN particles on various cell types in vitro, namely THLE-2 liver cells, HSC-T6 cells (rodent hepatic stellate cell line), and mouse lung cells (FIGS. 3A-3C) were tested. CCK-8 cell proliferation assay showed that MRINs promote the growth of THLE-2 liver cells (FIG. 3D) with a dose-responsive manner. In contrast, co-culture with MRINs suppressed the proliferation of HSC-T6 cells (FIG. 3E) and had no effects on mouse lung cells (FIG. 3F).

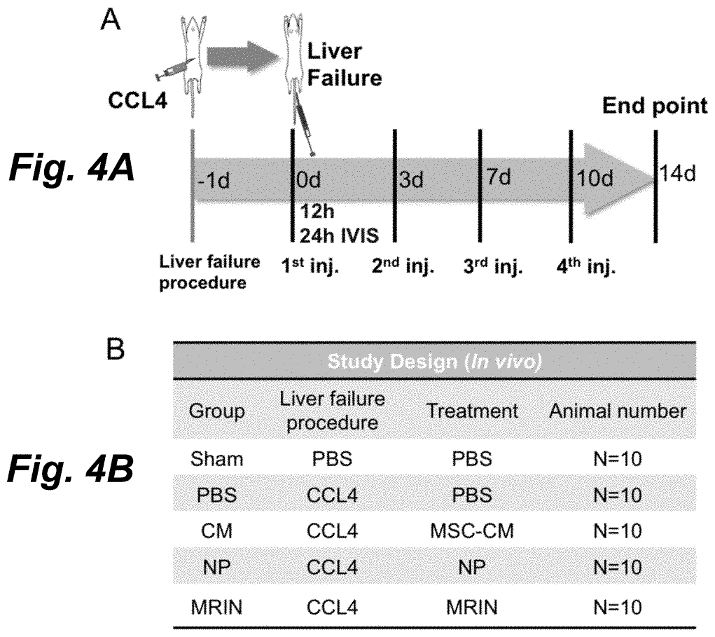

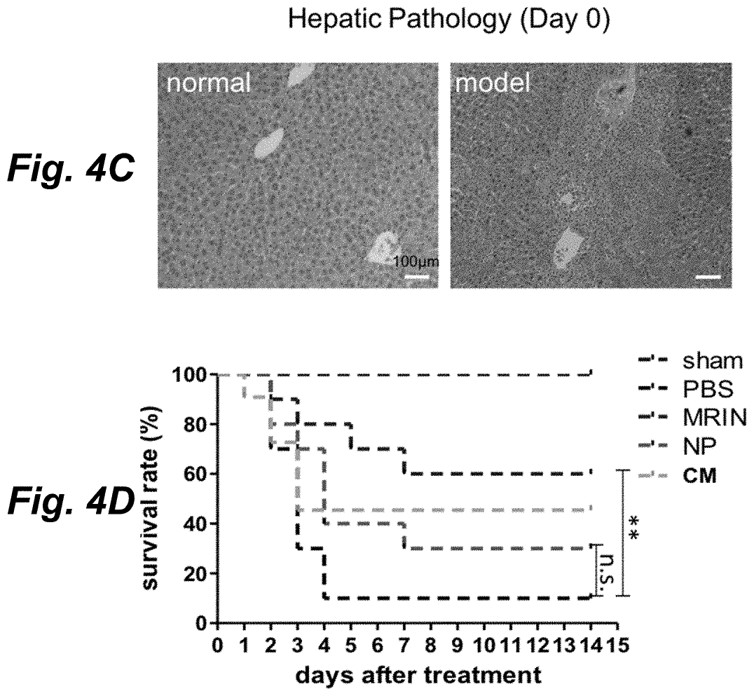

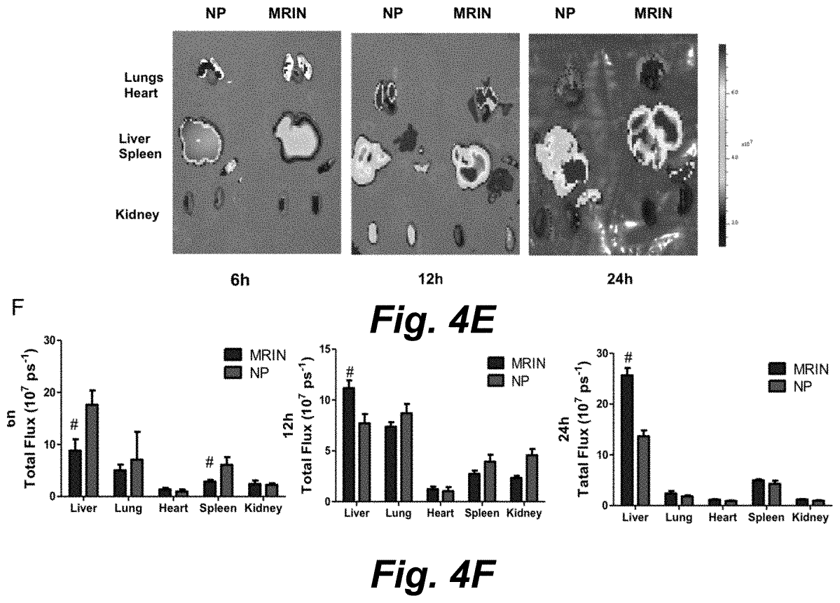

To test whether the RBC coat on MRINs increases the stability of the nanoparticles by discouraging macrophage uptake, M1 macrophage cells were co-cultured with control (non-coated NPs) or MRINs. After 3 hrs of co-incubation, approximately 80% of NPs were uptake by macrophages while only approximately 20% of MRINs were endocytosed (FIGS. 3G-3J). This was further confirmed by in vivo studies, indicating minimal internalization of MRINs by liver macrophage cells (CD45) after intravenous injection (FIG. 3K). Despite uptake by liver cells, a large amount of MRINs remained in the extracellular space (FIG. 3L). Biodistribution of MRINs in mice with liver failure: To test the therapeutic potential of MRINs, a mouse model of acute liver failure (FIGS. 4A and 4B) was used. Liver failure was evident in mice that received injections of carbon tetrachloride (FIG. 4C) as there was a large area of necrosis and inflammation in the hepatic lobule center. Over the two weeks, intravenous MRIN therapy significantly improved the survival of the animals while NP therapy and CM therapy only marginally improved the survival rate (FIG. 4D).

Twelve hours after intravenous injection, MRINs or NPs could be detected in the lungs, livers, spleens, and kidneys of the treated animals (FIG. 4E, middle). At 24 hrs, the signals in the lungs and kidneys decayed with increased signal in the livers (FIG. 4E, right). At either time point, more MRINs than NPs were detected in the liver, suggesting the RBC coating on MRINs enhanced liver retention (FIG. 4F).

To increase the temporal resolution of biodistribution, the organ distributions of MRINs and NPs were examined at an earlier time point (6 hrs). At 6 hrs, more NPs than MRINs were detected in liver, suggesting more rapid clearance of NPs by the liver macrophages. This is consistent with the in vitro data presented in FIGS. 3A-3L. In addition, the biodistribution of MRINs and NPs was examined in normal mice (instead of liver-failure mice). The organ pattern of biodistribution in normal mice was similar to that from liver-failure mice. At any given time point, the organ retention of MRINs in normal mice was similar to that in liver-failure mice.

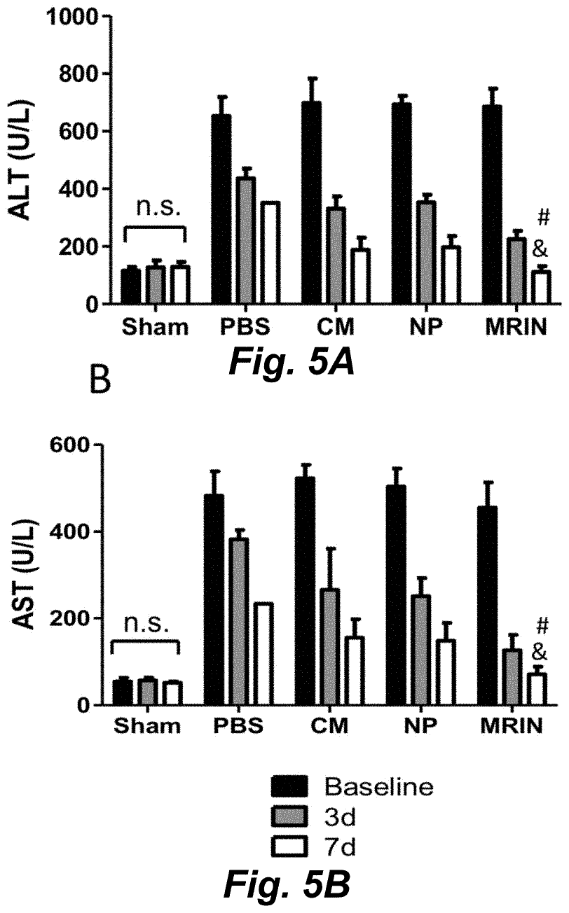

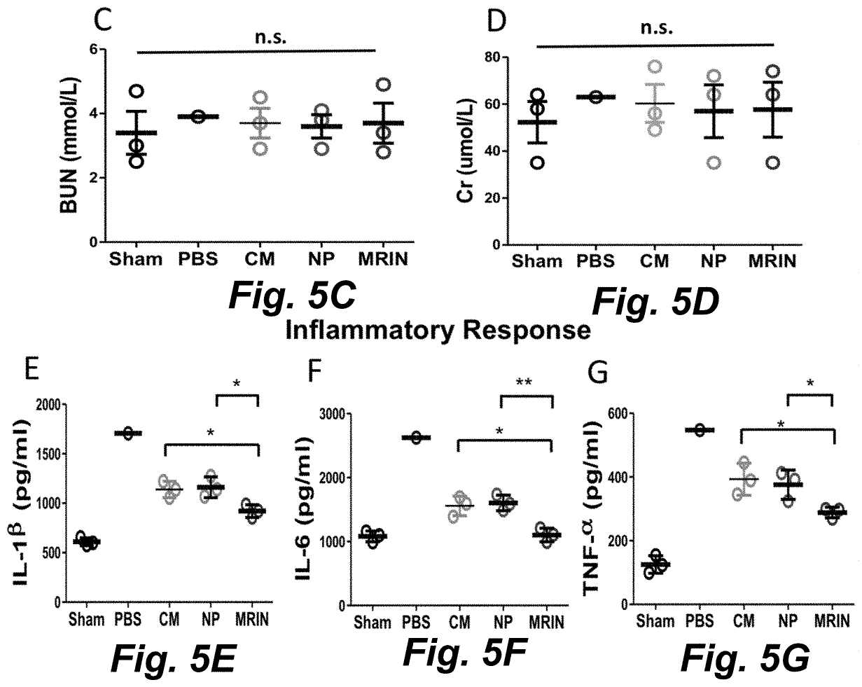

MRIN therapy protects liver functions in CCL4-induced liver failure: Alanine aminotransferase (ALT) and aspartate aminotransferase (AST) are biomarkers for liver functions. At baseline, the ALT and AST levels were identical among all treatment groups, indicating a similar degree of initial injury by carbon tetrachloride (FIGS. 5A and 5B, baseline).

At 3 d and 7 d post-therapy, MRIN injections significantly reduced ALT and AST levels. Injection of NP or CM resulted in a certain degree of liver function protection as compared to the PBS control group. However, MRIN injection generated a larger therapeutic benefit than the CM or NP group. Injection of MRIN particles did not impair the kidney functions of the mice (FIGS. 5C and 5D). MRIN therapy effectively reduced the circulation levels of proinflammatory cytokines such as interleukin-6 (IL-6), interleukin-1 beta (IL-1β), and tumor necrosis factor-α (TNF-α) in the animals (FIGS. 5E-5G), suggesting the anti-inflammatory role of MRIN therapy.

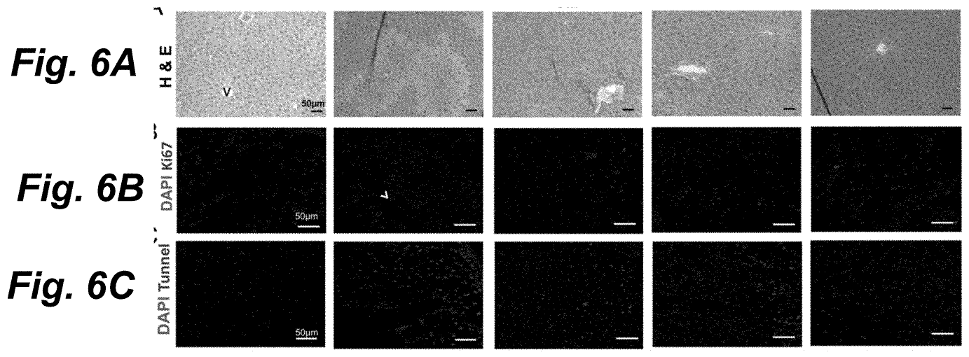

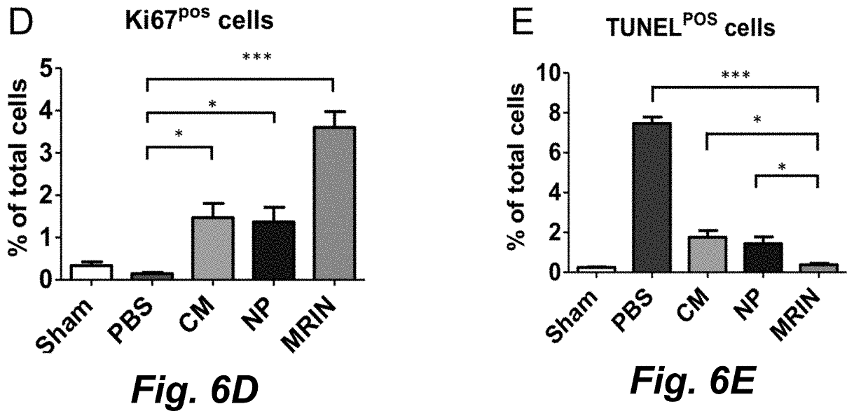

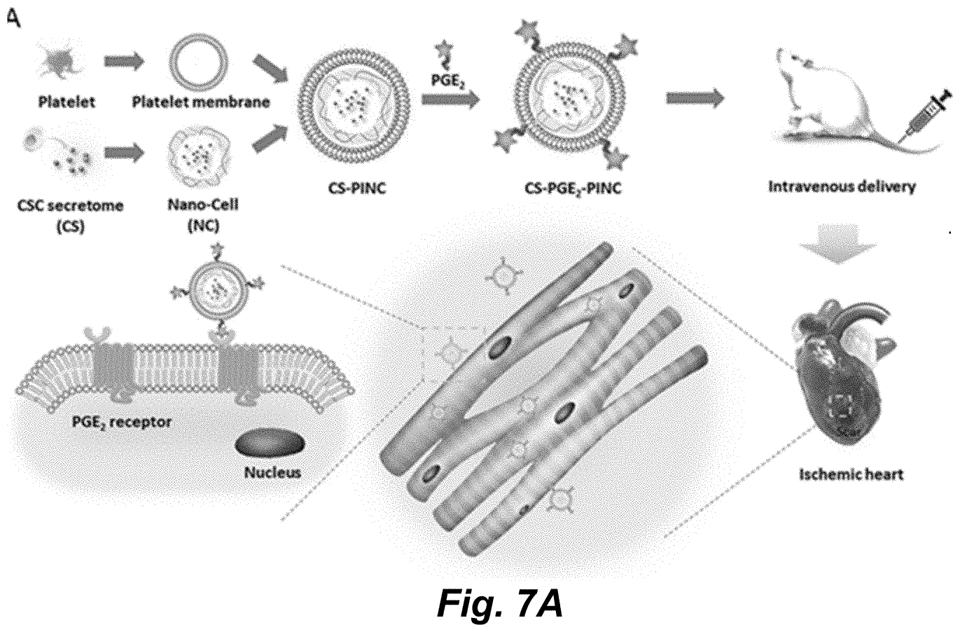

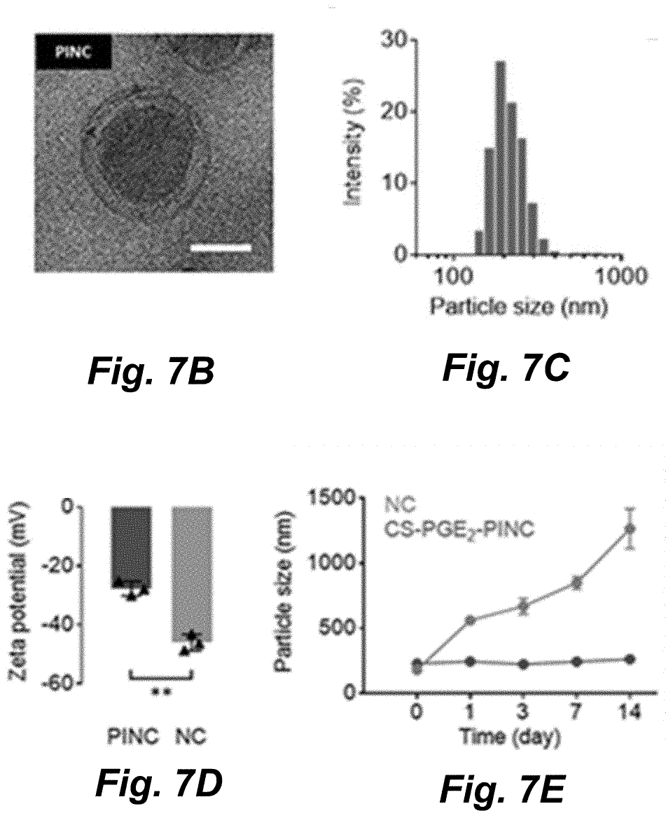

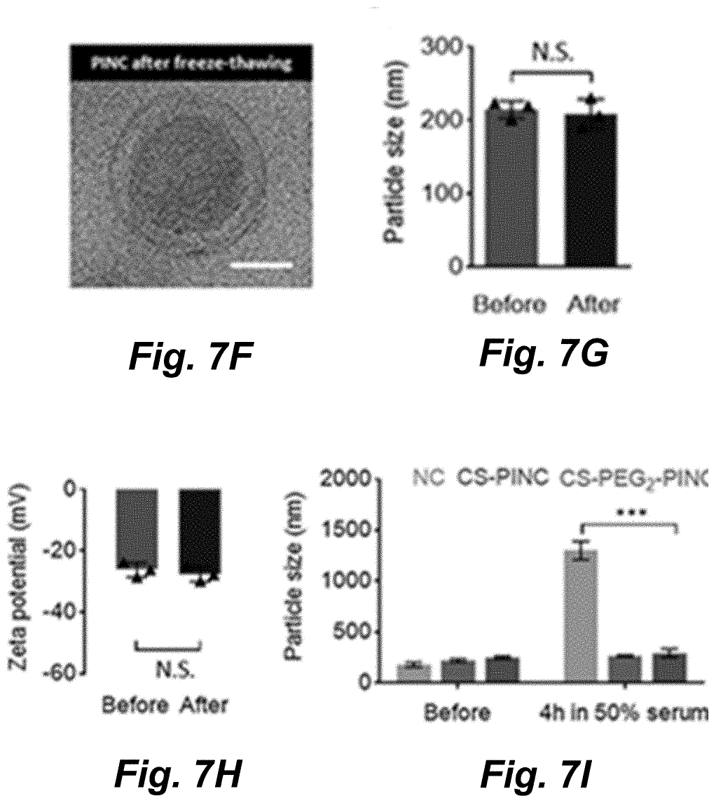

MRIN therapy reduces apoptosis and promotes regeneration: H&E staining indicated that MRIN therapy protected liver morphology (FIG. 6A). A sham group showed normal hepatic lobule structures and portal area. The control group exhibited the sign of hepatic lobule structure disorder and hepatocellular edema. NP or CM treatment led to a certain degree of recovery in hepatic lobule structures. However, there remained a degree of inflammatory cell infiltration in the portal area. MRIN treatment led to the preservation of normal hepatic lobule structures.

Ki67 staining showed that MRIN treatment promoted liver cell proliferation, suggesting higher levels of regeneration (FIGS. 6B and 6D). In addition, terminal deoxynucleotidyl transferase-mediated dUTP nick end labeling (TUNEL) staining showed that MRIN injection was more potent than CM or NP therapy in reducing apoptosis in CCL4-induced liver failure (FIGS. 6C and 6E).

The present disclosure encompasses compositions that combine red blood cell-derived membrane vesicles and a nanoparticle comprising all or a fraction of a secretome of cultured mesenchymal stem cells, including extracellular vesicles such as exosomes derived from the mesenchymal stem cells to harness the ability of red blood cell-derived membrane vesicles to afford protection to nanoparticles in the blood stream. Advantageously, this prolongs their efficacy and biodistribution in an animal or human recipient of the nanoparticles. Such decoration of nanoparticles comprising mesenchymal stem cell conditioned medium or extracellular vesicles such as exosomes derived from such as mesenchymal stem cells, with red blood cell-derived membrane vesicles is nontoxic as it does not alter the viability and functions of the extracellular vesicles.

The exosomes of the disclosure may be obtained by culturing an isolated population of mesenchymal stem cells whereupon the cells can secrete into the surrounding culture medium proteins, peptides, cytokines and the like, but also secreted microparticles or exosomes that may encapsulate proteins, miRNA, and the like to form the cell secreteome

The membrane vesicles of the disclosure comprise fragments of the membranes of red blood cells (erythrocytes) and accordingly comprise a spectrum of the cell surface markers of the parent RBCs and, in particular, those ligands or receptor proteins or peptides that allow MRINs to be recognized as hemocompatable. The methods of generating the engineered stem cells and their use, as herein disclosed, may also be usefully applied to any suitable stem cells for the regeneration of tissues other than cardiac tissue.

The present disclosure, therefore, provides methods for the generating of populations of RBC membrane coated mesenchymal stem cell-derived nanoparticles for delivery to a tissue injury site to most advantageously be used for administration to subject having damaged or diseased tissue so as to repair, regenerate, and/or improve the anatomy and/or function of the damaged or diseased tissue. While multiple types of stem cells may be processed according to the methods disclosed herein, in several embodiments, stem cells such as, but not limited to, mesenchyme stem cell secretomes can be usefully generated by processing tissue and then engineered to target a site of disease or injury. The mesenchymal stem cell secretome can be fused with RBC-derived membrane vesicles such that RBC-specific cell surface components are included in the outer MRIN membranes of the stem cells. Alternatively, extracellular vesicle such as an exosomes derived from cultured mesenchymal stem cells can be encapsulated by the RBC-derived membrane vesicles. The MRINs of the disclosure can concentrate at the site of hepatic injury and increases the likelihood of establishing regeneration of or liver injury or reducing the effects of the injury or disease.

Provided herein, therefore, are methods of treating a patient with an injured organ such as the liver by administering the mesenchymal stem cell-derived nanoparticles, as described herein.

Therapy (and the methods disclosed herein) using the MRINs of the disclosure may be autologous, allogeneic, syngeneic, or xenogeneic, depending on the needs of the subject patient. In several embodiments, allogeneic therapy is employed, as the ready availability of tissue sources (e.g., organ donors, etc.) enables a scaled-up production of a large quantity of cells that can be stored and subsequently used in an “off the shelf” fashion. Preferably, the source of both the stem cells (or extracellular vesicle such as an exosomes thereof) and the RBCs that provide the membrane vesicles is the subject patient that receives the MRINs to reduce the possibility of adverse immunological reactions. However, it may also be advantageous to use stored mesenchymal stem cells and RBCs derived from another person when the subject patient requires treatment earlier than the time required to culture his/her own mesenchymal stem cells for fusion with RBC membrane vesicles.

Methods of administration include injection, transplantation, or other clinical methods of getting cells to a site of injury in the body. Non-limiting examples of injection methods that can be used to administer MRINs of the disclosure include intravenous injection, intracoronary injection, transmyocardial injection, epicardial injection, direct endocardial injection, catheter-based transendocardial injection, transvenous injection into coronary veins, intrapericardial delivery, or combinations thereof.

The injection of MRINs of the disclosure can be either in a bolus or in an infusion. The engineered MRINs of the disclosure can be combined with a pharmaceutical carrier suitable for administering to a recipient subject. Pharmaceutically acceptable carriers are determined in part by the particular method used to administer the cell composition, but are typically isotonic, buffered saline solutions. Accordingly, there are a wide variety of suitable formulations of pharmaceutical compositions for the presently described compositions (see, e.g., Remington's Pharmaceutical Sciences, 17th ed., 1989). The engineered cardiac stem cells of the disclosure as described herein can be administered in a single dose, a plurality of doses, or on a regular basis (e.g., daily) for a period of time (e.g., 2, 3, 4, 5, 6, 7, days, weeks, months, or as long as the condition persists).

The dose (e.g., the amount of cells) administered to the subject in the context of the present disclosure should be sufficient to affect a beneficial response in the subject over time, e.g., repair or regeneration of heart tissue, repair of regeneration of blood vessels, or a combination thereof. The optimal dose level for any patient will depend on a variety of factors including the efficacy of the specific modulator employed, the age, body weight, physical activity, and diet of the patient, on a possible combination with other drugs, and on the severity of the cardiac or angiogenic injury. The size of the dose also will be determined by the presence, existence, nature, and extent of any adverse side-effects that accompany the administration of the engineered cardiac stem cells in a particular subject.

Vascular endothelium provides a barrier between subendothelial matrix and circulating cells including blood cells and platelets. It has been established that ischemic heart injures such as acute myocardial infarction (MI) can induce vascular damage and expose components of subendothelial matrix including collagen, fibronectin and von

Willebrand factor (vWF) to recruit platelets. Platelets can then accumulate and bind to the injured vasculatures in MI. Such platelet recruitment is based on the matrix-binding abilities of various platelet surface molecules such as glycoprotein (GP) VI, GPIb-IX-V and gpIIb/IIIa (Lippi et al., (2011) Nat. Rev. Cardiol. 8: 502-512). Platelets may form co-aggregates with circulating CD34⁺ progenitors in patients with acute coronary syndromes and thereby increase peripheral recruitment within the ischemic microcirculatory district and promote adhesion to the vascular lesion to promote healing (Stellos et al., (2013) Eur. Heart J. 34: 2548-2556). Cardiosphere-derived cardiac stem cells (or CSCs) have been investigated, from laboratory animal model studies (Li et al., (2012) J. Am. Coll. Cardiol. 59: 942-953; Smith et al., (2007) Circulation 115: 896-908; Cheng et al., (2010) Circ. Res. 106: 1570-1581; Lee et al., (2011) J. Am. Coll. Cardiol. 57: 455-465; Cheng et al., (2014) JACC Heart Fail. 2: 49-61) to a recently completed phase I clinical trial (Malliaras et al., (2014) J. Am. Coll. Cardiol. 63: 110-122; Makkar et al., (2012) Lancet 379: 895-904), for the treatment of MI. Like other cell types, CSCs also suffer a deficit retention rate in the heart after delivery (Cheng et al., (2014) Nat. Commun. 5: 4880).

The present disclosure encompasses compositions that combine platelet-derived membrane vesicles and cardiac stem cell-derived nanoparticles comprising at least one cardiac stem cell secreted factor, a population of cardiac stem cell-derived exosomes, or cardiac stem cell-derived conditioned medium encapsulated in a biodegradable polymer matrix or shell to harness the ability of platelets, or the membranes thereof, to target a site of injury to a blood vessel. Stem cells such as cardiac or mesenchymal stem cells offer the advantage of the ability to invade and differentiate into cell types for the repair of injured tissue. Such decoration of cells, or extracellular vesicles such as an exosomes derived from such cells, with platelet-derived membrane vesicles is nontoxic as it does not alter the viability and functions of extracellular vesicles, but augments the targeting of the exosomes for enhanced therapeutic outcomes by selectively binding to the subendothelial matrix.

The present disclosure also encompasses embodiments of a method for generating a population of nanoparticles comprising extracellular vesicle such as stem cell-derived exosomes derived therefrom that can specifically target injury-exposed ligands or receptors of platelet-specific polypeptide markers. In particular, the methods of the disclosure are advantageous for the generation of microparticles that, when administered to a subject having a tissue injury, will specifically target and bind to injured vascular tissue. While the methods of cell or extracellular vesicle engineering disclosed can be applied to any cell line, and in particular, to stem cells, or exosomes derived therefrom, it had been found especially advantageous to engineer cardiac stem cells or their exosomes to target the site of cardiac or cardiovascular injury.

The present disclosure encompasses the ability of platelet surface markers to selectively bind to a site of subendothelial matrix following the denuding of the endothelial cells after a vascular injury event. For example, myocardial infarction, ischemic stroke, and the like result in vascular damage that indirectly leads to injury or death to tissues normally sustained by the blood vessel. Particularly vulnerable is cardiac tissue, brain or other neural tissue. Loss of vascular endothelial cells exposes sites of the underlying matrix that allow the platelet-binding sites fused into the membranes of stem cells to selectively bind thereto to concentrate the engineered stem cells at the site of injury. The attached cells may then migrate through the matrix into the surrounding damaged tissue, whereupon the stem cells can differentiate, proliferate, and thus regenerate the lost or injured or lost tissue.

The stem cells of the disclosure are an isolated population of cells that may be expanded by tissue culture after being isolated from a tissue of an animal or human. Most advantageously the cells are, but not limited to, stem cells such as stem cells isolated from cardiac tissue, cultured under conditions appropriate for population expansion as cardiospheres and then incubated with platelet membrane vesicles.

These membrane vesicles consist of fragments of the outer membranes of platelets and accordingly comprise a spectrum of the cell surface markers of the parent platelets and, in particular, those ligands or receptor proteins or peptides that allow platelets to bind to such as the subendothelial matrix of a blood vessel or cardiac tissue exposed by injury, for example, due to a myocardial infarction such a myocardial. The methods of generating the microparticles and their use, as herein disclosed, may also be usefully applied to any suitable stem cells for the regeneration of tissues other than cardiac tissue.

The microparticles of the disclosure can be fused with platelet-derived membrane vesicles such that platelet-specific cell surface components are included in the outer cell membranes of the stem cells. Advantageously, the platelet-derived membrane shell of the microparticles may be further modified by attaching thereto a ligand or ligands known to specifically bind to a biomarker on the surface of a targeted cell type or tissue. For, example, but not intended to be limiting, prostaglandin E2 (PGE2) may be conjugated to the platelet-derived membrane shell to specifically target cells in the recipient subject that have PGE2 cell-surface receptors. Targeting the site of cardiac or blood vessel damage concentrates microparticles at the injury and increases the likelihood of establishing regeneration of damaged cardiac or cardiovascular injury.

Multiple types of cardiac stem cells can be obtained according to the methods disclosed herein including, but not limited to, cardiospheres and cardiosphere-derived cells (CDCs), ckit (CD117)-positive cells, nkx2.5-positive cardiac cells, and the like. Embodiments of the engineered cardiac stem cells of the present disclosure are advantageous for the treatment or repair of damaged or diseased cardiac tissue that may have resulted from one or more of acute heart failure (e.g., a stroke or myocardial infarction) or chronic heart failure (e.g., congestive heart failure). In some embodiments of the methods of the disclosure, about 1×10⁵ to about 1×10⁷ of cardiac stem cells may be administered. The dose can be varied depending on the size and/or age of a subject receiving the cells. Different routes of administration are also used, depending on the embodiment. For example, the cardiac stem cells may be administered by intravenous, intra-arterial, intracoronary, or intramyocardial routes of administration.

Provided herein, therefore, are methods of treating a human subject with an injured heart by administering cardiac stem cell-derived microparticles, as described herein. In some embodiments, the microparticles may be administered to a patient with acute myocardial infarction or having myocardial ischemia. Further provided are methods of treating a patient in need of angiogenesis (e.g., growth of blood vessels) by administering the cardiac stem cell-derived particles of the disclosure. The cardiac stem cell-derived microparticles of the disclosure can be used to ameliorate the effects of any type of injury to the heart.

Cardiac stem cell-derived microparticle therapy (and the methods disclosed herein) may be autologous, allogeneic, syngeneic, or xenogeneic, depending on the needs of the subject patient. In several embodiments, allogeneic therapy is employed, as the ready availability of tissue sources (e.g., organ donors, etc.) enables a scaled-up production of a large quantity of cells that can be stored and subsequently used in an “off the shelf” fashion. Preferably, the source of both stem cells (or extracellular vesicle such as an exosomes thereof) and the platelets that provide the membrane vesicles is the subject patient that receives the engineered cells or extracellular vesicles to reduce the possibility of adverse immunological reactions. However, it may also be advantageous to use stored stem cells and platelet-rich plasma derived from another person when the subject patient requires treatment earlier than the time required to culture his/her own cardiac stem cells for fusion with platelet membrane vesicles.

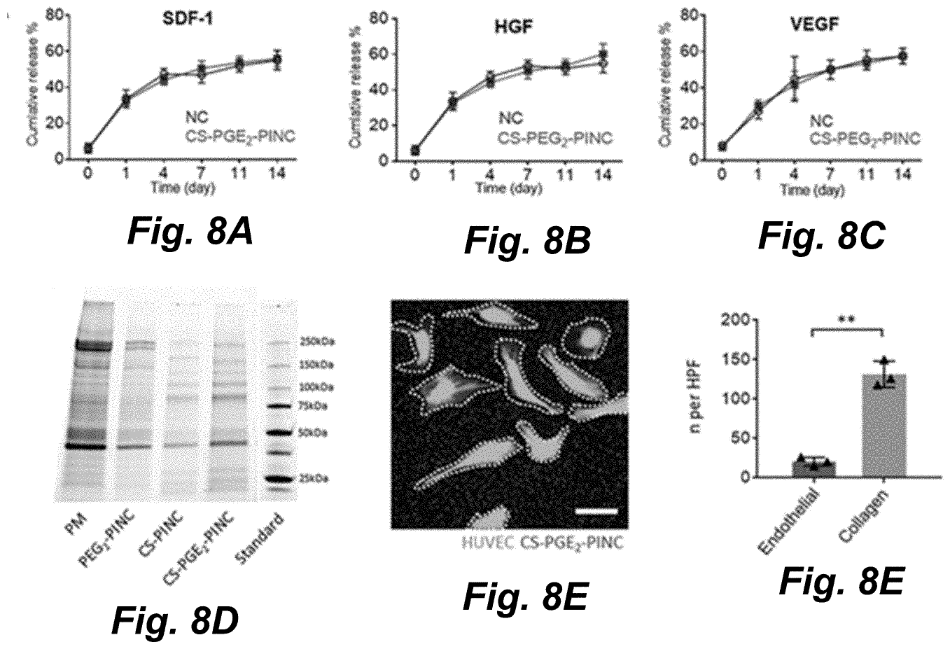

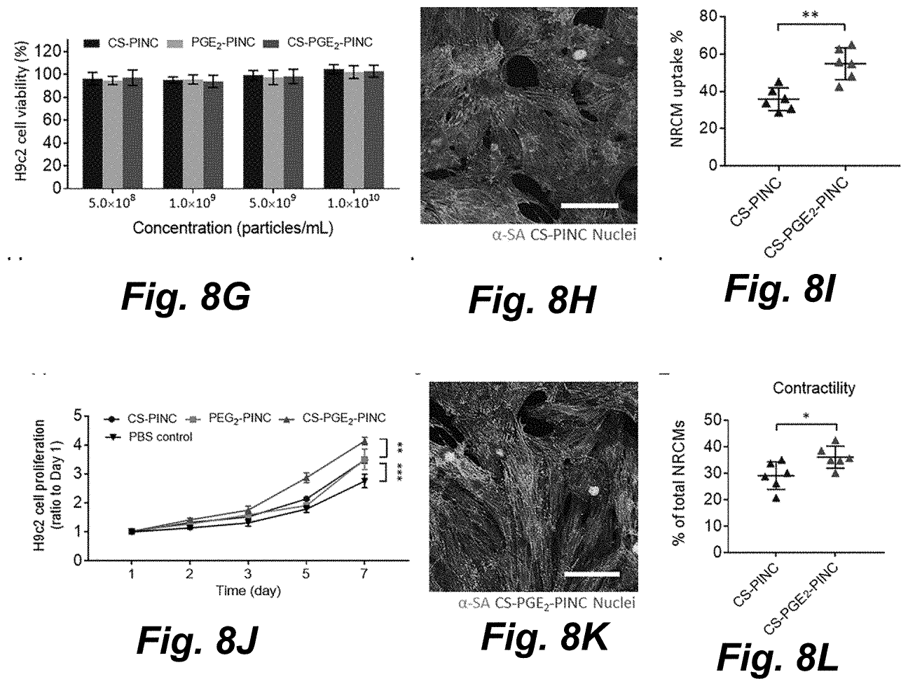

Accordingly, further provided by the disclosure is a platelet-inspired nano-cell (PINC) that comprises a CSC core and a platelet membrane shell. The CSC core comprises a CSC secretome encapsulated in a polymer such as, but not limited to a poly (lactic-co-glycolic acid) (PLGA), to form a nanoparticle. The platelet membrane enveloping the nanoparticle core may be modified by such as having PGE₂ conjugated thereto and which is expected to both enhance the cardiomyocyte targetability and facilitate the endogenous repair through PGE₂/EP receptor signaling after I/R injury (FIG. 8A). As a novel biomimetic therapeutic nanoparticle, PINC of the disclosure can offer the following advantages compared to natural stem cells: (i) systemic administration: the nano size of PINC enables intravenous application; unlike stem cells, PINCs are less likely to clog the lungs; (ii) dual targeting: the platelet membrane on PINCs targets injured blood vessels while the PGE₂ targets injured cardiomyocytes in I/R; and (iii) stability: unlike real stem cells, PINCs can be used in an off-the-shelf fashion since there are no living components in it.

Synthesis and Characterization of PINCs: Double-emulsion-based solvent evaporation/extraction was combined a with cell membrane cloaking technique to prepare the PINCs (FIG. 8A). First, the secretome derived from CSCs was incorporated into PLGA to form nano-cells (NCs) through a double emulsion method. The CSC secretome loading capacity and efficiency were 2.8 wt % and 85.3%, respectively, showing that the CSC secretome was efficiently encapsulated into the hydrophilic core of NCs. The platelet membrane was then isolated and purified from the platelet-rich plasma through gradient centrifugation.^{[42]} To prepare the PINC functionalized with PGE₂, the amine groups of platelet membrane glycoprotein were further reacted with the terminal carboxyl group of PGE₂ based on N-Ethyl-N′-(3-dimethylaminopropyl)carbodiimide (EDC)/N-hydroxysuccinimide (NHS) activation to obtain the PGE₂-platelet membrane conjugate. The content of PGE₂ conjugated to the PGE₂-platelet membrane conjugate was determined to be 0.68 mg g⁻¹ dry platelet membrane by enzyme-linked immunosorbent assay (ELISA), with a high conjugation yield of over 95% (FIG. 14). The conjugation of PGE₂ onto the surface of platelet membrane was validated by confocal laser scanning microscopy (CLSM). The colocalization of the fluorescence signals from Dil-labeled platelet membrane (red) and fluorescein isothiocyanate-tagged PGE₂ (green) substantiated the successful conjugation of PGE₂ onto the platelet membrane surface (FIG. 1). The resulting PGE₂-platelet membrane conjugate was subsequently incubated with the CSC secretome-loaded NC under ultrasonication, followed by membrane extrusion to form the PGE₂-platelet-membrane-coated NC (designated CS-PGE₂-PINC). The PINC functionalized with only CSC secretome (designated CS-PINC) was prepared via coating the purified platelet membrane on the surface of NC. The PINC functionalized with only PGE₂ (designated PGE₂-PINC) was prepared via coating the PGE₂-platelet membrane conjugate on the surface of bare PLGA nanoparticle. Transmission electron microscopy (TEM) studies confirmed the platelet membrane coating on the CS-PGE₂-PINCs that appear as a core-shell structure (FIG. 8B). Dynamic light scattering (DLS) analysis indicated that the CS-PGE₂-PINCs had an average diameter of about 195 nm and a narrow size distribution (polydispersity index (PDI)=0.157) (FIGS. 8B and 8C). Nanoparticle tracking analysis using NanoSight revealed that the majority of particles showed a particle size of about 205 nm, consistent with the results obtained from TEM and DLS measurements (FIGS. 16A and 16B).

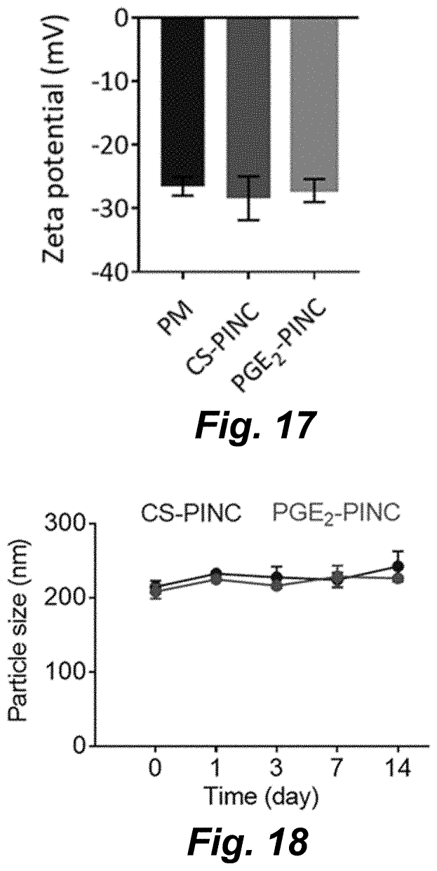

After cloaking with platelet membrane, the size of PINCs did not change significantly compared to the bare NC (FIG. 17) while the zeta potential of CS-PGE₂-PINC increased by about 18 mV compared with bare NC, approaching the value of −27 mV (FIGS. 8D and 18). This phenomenon is consistent with the previously reported nanoparticles after platelet membrane coating, which can be ascribed to the veiling of the highly negative PLGA core with the less negatively charged platelet membrane (Wang et al., (2016) Adv. Funct. Mater. 26: 1628; Hu et al., (2016) Adv. Mater. 28: 9573; Hu et al., (2015) Nature 526: 118).

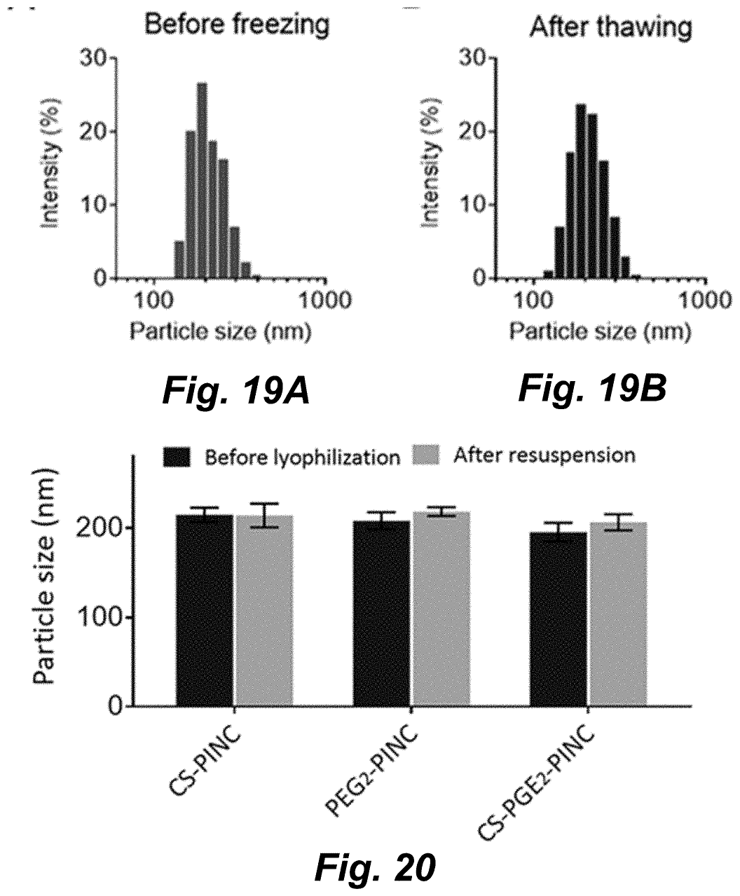

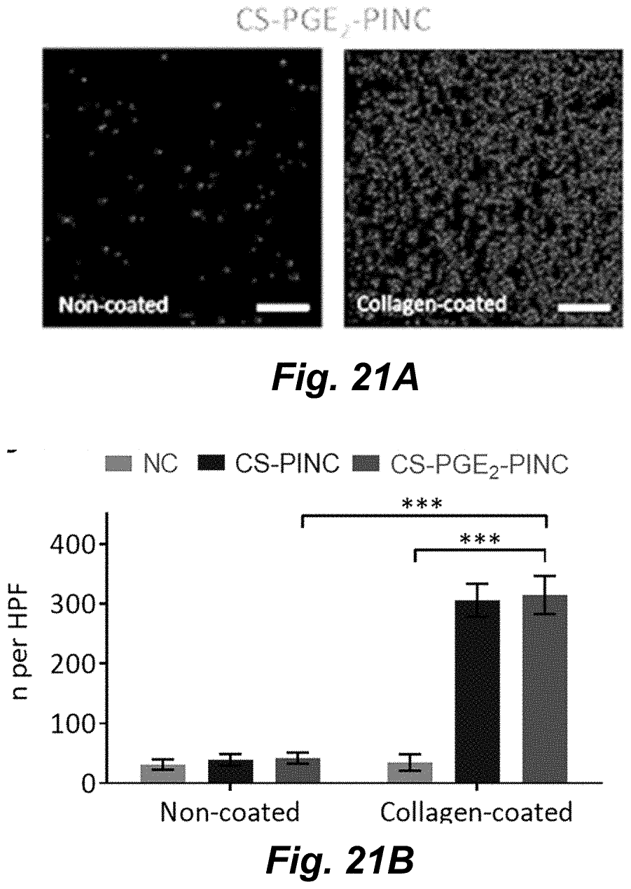

To determine the stability of different nanoformulations in solution over time, NCs and CS-PGE₂-PINCs were stored in phosphate buffered saline (1×PBS, pH 7.4) at room temperature, respectively, and their size change was monitored by DLS. The PINCs exhibited stable size over a 2-week study period, while the NCs showed rapidly agglomeration in PBS (FIGS. 8E and 19). In addition, the cloaking of platelet membrane endowed the PINCs with superior stability before and after incubation in 50% serum when compared with bare NC (FIG. 8I). The negatively-charged cell-mimicking surface and superior serum stability make the PINCs ideal for intravenous application. The long-term storage stability of CS-PGE₂-PINCs was investigated. After cryopreservation for over 3 months, the CS-PGE₂-PINCs after thawing exhibited similar morphology, size, and surface charge to those before freezing (FIGS. 8F-8H, 20A, and 20B). Furthermore, all the PINC formulations exhibited excellent lyophilization stability, with the size and zeta potential remained nearly identical before lyophilization and after resuspension (FIG. 21).

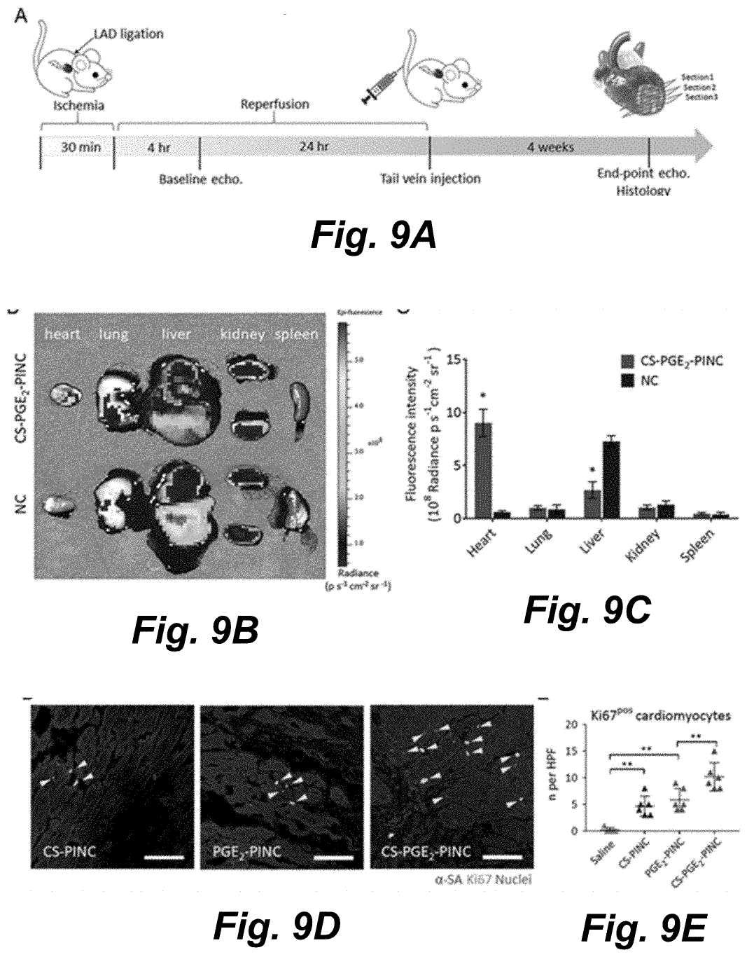

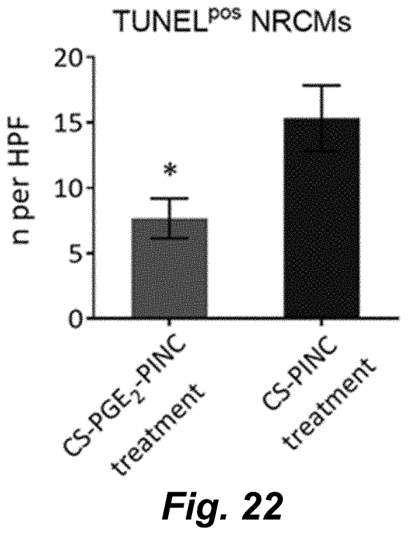

Bioactivity of PINCs: Stem cell therapy represents a promising strategy for treating ischemic heart diseases (Menasché et al., (2016) Eur. Heart J. 36: 2011). Mounting lines of preclinical and clinical evidence indicate that stem cells, including CSCs and MSCs, exert their functional benefits through the secretion of paracrine factors, acting like “mini-drug pumps” to promote endogenous repair (Li et al., (2012) J. Am. Coll. Cardiol. 59: 942; Barry & Murphy: (2013) Nat. Rev. Rheumatol. 9: 584). Fabricated therapeutic cell-mimicking microparticles by packaging stem cell factors in a biodegradable polymeric shell and their regenerative potential in rodent models of heart injury have been described (Luo et al., (2017) Circ. Res. 120: 1768; Tang et al., (2017) Nat. Commun. 8: 13724). It has now been shown whether our PINCs could mimic CSCs by secreting regenerative growth factors. ELISA revealed that the CS-PGE₂-PINCs continuously released pro-myogenic and pro-angiogenic paracrine factors, such as stromal cell-derived factor-1 (SDF-1), vascular endothelial growth factor (VEGF), and hepatocyte growth factor (HGF) for at least 14 days; the platelet membrane coating did not affect the release of stem cell factors from PINCs (FIGS. 9A-9C). SDS-PAGE was used to run platelet membrane and all the different PINCs for protein composition analysis. As expected, all the PINCs had protein profiles that are similar to that of the platelet membrane, further confirming the successful platelet membrane coating onto PINCs (FIG. 9D). Owing to the platelet-mimicking properties, the PINCs showed robust binding to the collagen-coated surface (FIGS. 22A and 22B). In addition, Dil-labeled PINCs were plated onto green fluorescent protein-tagged human umbilical vein endothelial cells (GFP-HUVECs) cultured on collagen-coated surface and the selective adherence of PINCs to the collagen region was confirmed (FIGS. 9E and 9F).

The effect of the PINCs on the H9c2 cardiomyoblasts, a widely-used cardiac cell line isolated from the embryonic rat heart tissue was tested. The CS-PINCs, PGE₂-PINCs, and CS-PGE₂-PINCs have excellent cytocompatibility as confirmed by the cell viability assay. The cells maintain high viability upon exposure to the PINCs (>95% viability), regardless of nanoparticle concentration (FIG. 9G). In addition, cell proliferation assay using a cell count kit-8 (CCK-8) revealed that PINCs promote the growth of H9c2 cardiomyoblasts, indicating that the release of therapeutic stem cell factors from the PINCs promotes cell attachment and proliferation, consistent with our previous studies (Tang et al., (2018) Nat. Biomed. Eng. 2: 17; Luo et al., (2017) Circ. Res. 120: 1768; Tang et al., (2017) Nat. Commun. 8: 13724; Tang et al., (2017) ACS Nano 11: 9738). The H9c2 cells treated with CS-PGE₂-PINCs exhibited significantly higher proliferative potential than those treated with CS-PINCs or PGE₂-PINCs (FIG. 9H).

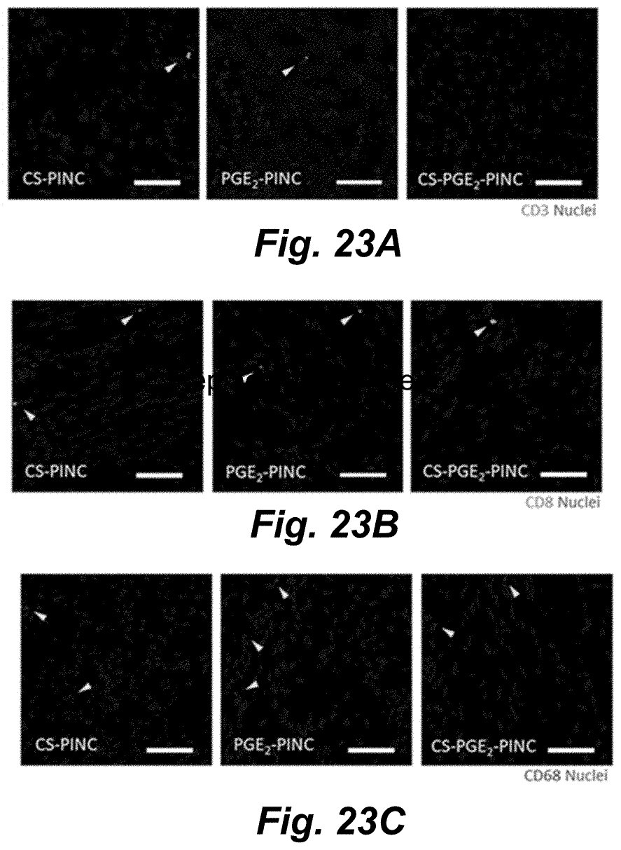

The effect of PGE₂ decoration on the cardiomyocyte protective ability of PINCs in vitro was investigated. Neonatal rat cardiomyocytes (NRCMs) were cocultured with Dil-labeled CS-PINCs or CS-PGE₂-PINCs (FIGS. 9I and 9J) with equivalent concentrations. After co-culturing for 3 h, the uptake of CS-PGE₂-PINCs into NRCMs (stained by α-sarcomeric actinin (α-SA), green) was significantly higher than that of the nanoparticles without PGE₂ decoration (FIG. 9K). Furthermore, CS-PGE₂-PINCs significantly promoted NRCM contractility compared with CS-PINCs (FIG. 9L). Following exposure to serum-free medium supplemented with hydrogen peroxide (250 μM) for 3 h, which simulates an ischemic microenvironment, TUNEL staining showed that the NRCMs pretreated with CS-PGE₂-PINCs were less apoptotic than those pretreated with CS-PINCs (FIG. 23). Together, these results suggest the enhanced heart-targeting ability and regenerative potential of CS-PGE₂-PINCs relative to CS-PINCs, which could be attributable to the specific interactions between CS-PGE₂-PINCs and the PGE₂ receptors expressed on cardiomyocyte or cardiomyoblast surface (Hsueh et al., (2014) EMBO Mol. Med. 6: 496; Li et al., (2016) Sci. Rep. 6: 36949).

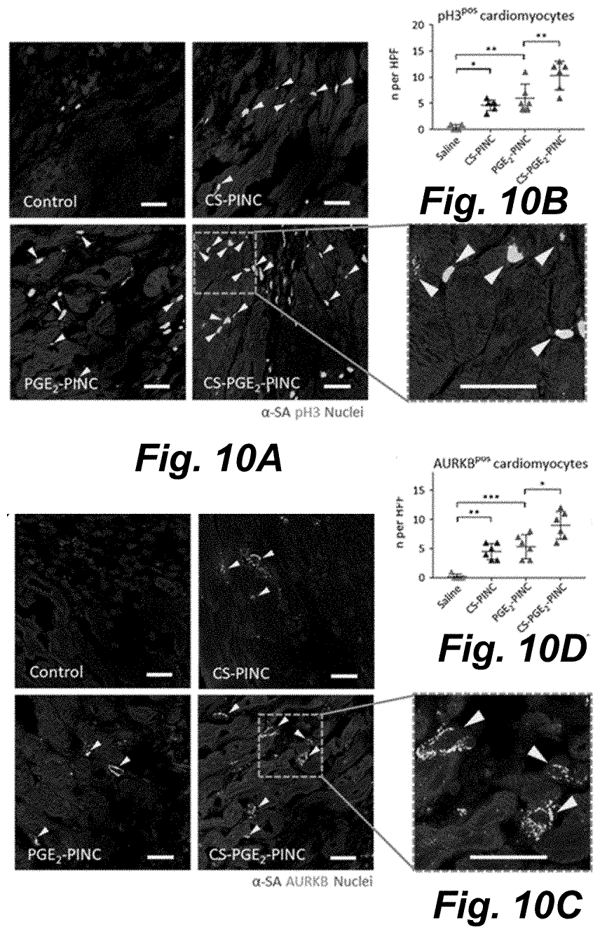

In Vivo Heart Targeting and Bioactivity of PINCs in Mice with Myocardial I/R Injury: Myocardial reperfusion therapy restores blood flow and is the current standard treatment for patients after a heart attack (Vicencio et al., (2015) J. Am. Coll. Cardiol. 65: 1525). However, it paradoxically causes further lethal tissue injury, known as myocardial I/R injury in clinical practice. To control or attenuate I/R injury is of clinical interest for improving post-ischemic recovery; thus the bioactivity of PINCs in immunocompetent CD1 mice with I/R injury was tested (FIG. 10A). Following a temporary left anterior descending coronary artery (LAD) ligation for 30 min to create ischemia injury and a subsequent 24-h reperfusion, the mice were randomly divided into four groups and treated with saline (negative control), CS-PINCs, PGE₂-PINCs, and CS-PGE₂-PINCs via tail vein injection.

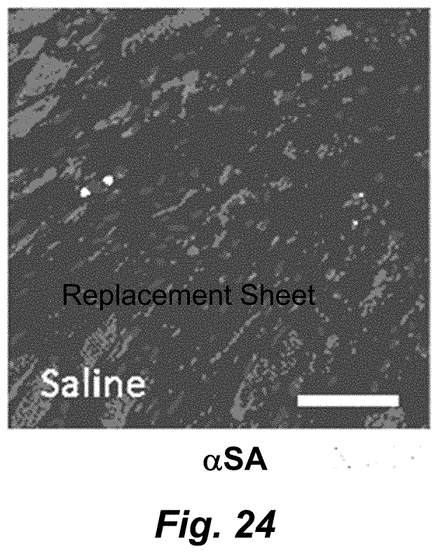

To evaluate the heart targeting capability of PINC, the mice intravenously administrated with DiR-labeled CS-PGE₂-PINCs or bare NCs following myocardial I/R injury were autopsied after 14 days to collect major organs for ex vivo fluorescent imaging. The infarcted hearts that received CS-PGE₂-PINCs exhibited stronger fluorescent signal than other organs as well as the hearts that received NCs (FIG. 10B). The quantitative region-of-interest (ROI) analysis confirmed that the CS-PGE₂-PINC-recipient hearts showed 14.9-fold higher fluorescence intensity than those treated with bare NCs, as well as 3.4-fold and 8.6-fold higher than the liver and kidney, respectively, validating the notable heart targeting ability of CS-PGE₂-PINCs (FIG. 10C). In contrast, greater nanoparticle accumulation was observed in the livers of animals that received the bare NCs compared to other organs, suggesting significant clearance of nanoparticles by the liver macrophages as expected. The biocompatibility of PINCs was evaluated. Negligible T-cell and macrophage infiltration were confirmed by the presence of few CD3-/CD8-positive T cells and CD68-positive macrophages in the hearts that received different PINCs, indicating good biocompatibility of these nanoformulations (FIGS. 24A-24C).

It has been established that adult cardiomyocytes have extremely limited capacity to proliferate in vivo. To test the bioactivity of PINCs in adult mouse cardiomyocytes, the cardiomyocyte proliferation was assessed 4 weeks after treatment by α-SA and Ki67 expressions. The number of Ki67-positive cardiomyocytes in the peri-infarct region of both the CS-PINC-recipient and the PGE₂-PINC-recipient hearts was significantly higher than that of the control hearts treated with saline injection, although the difference between the two groups was indiscernible (FIGS. 10D and 25). Notably, the highest number of cycling cardiomyocytes was found in the peri-infarct region of the CS-PGE₂-PINC-recipient hearts among all the groups (FIGS. 10D and 10E). The hearts were further stained for a specific marker of late G2/mitosis, phosphorylated histone H3 (pH3), and a marker of cytokinesis,

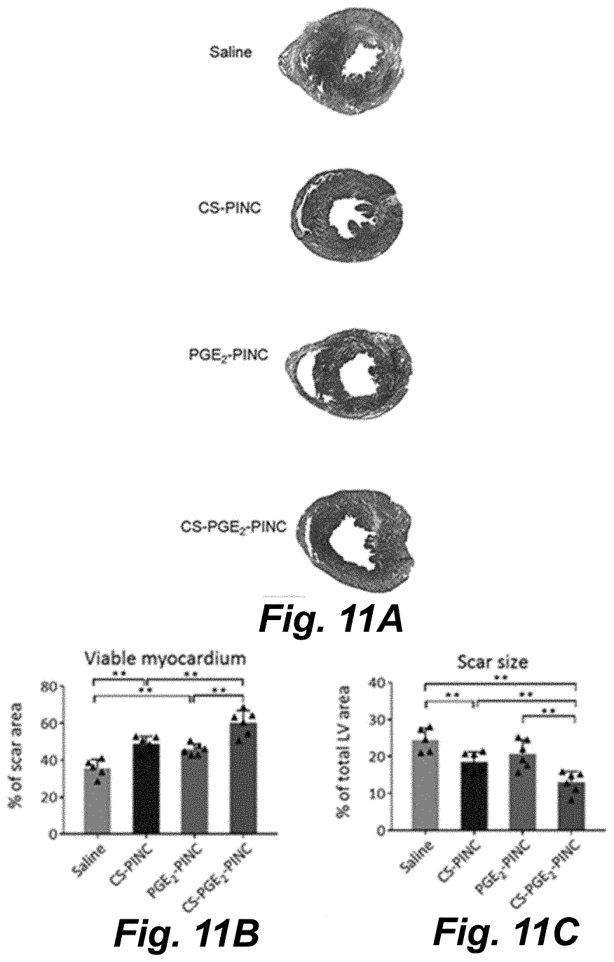

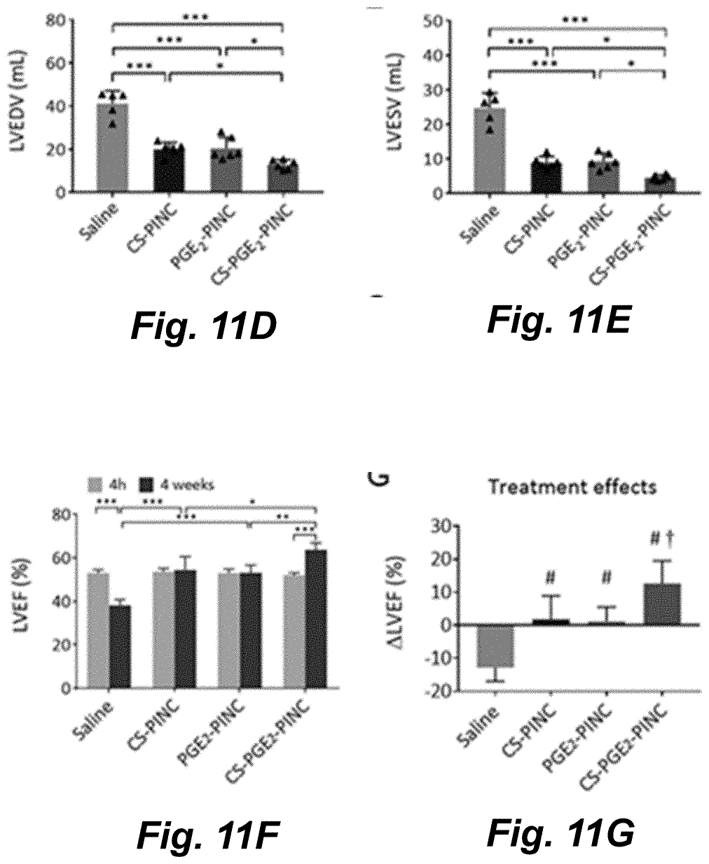

Aurora B kinase (AURKB). Remarkably, CS-PGE₂-PINCs induced robust mitotic activity of cardiomyocytes in the injured hearts after 4 weeks of treatment as evidenced by the elevated number of pH3-positive cardiomyocytes at the peri-infarct zone compared to other PINC treatment (FIGS. 11A and 11B). Expression of AURKB suggested that the cardiomyocytes not only entered the cell cycle but also were undergoing cytokinesis (FIGS. 11C and 11D).

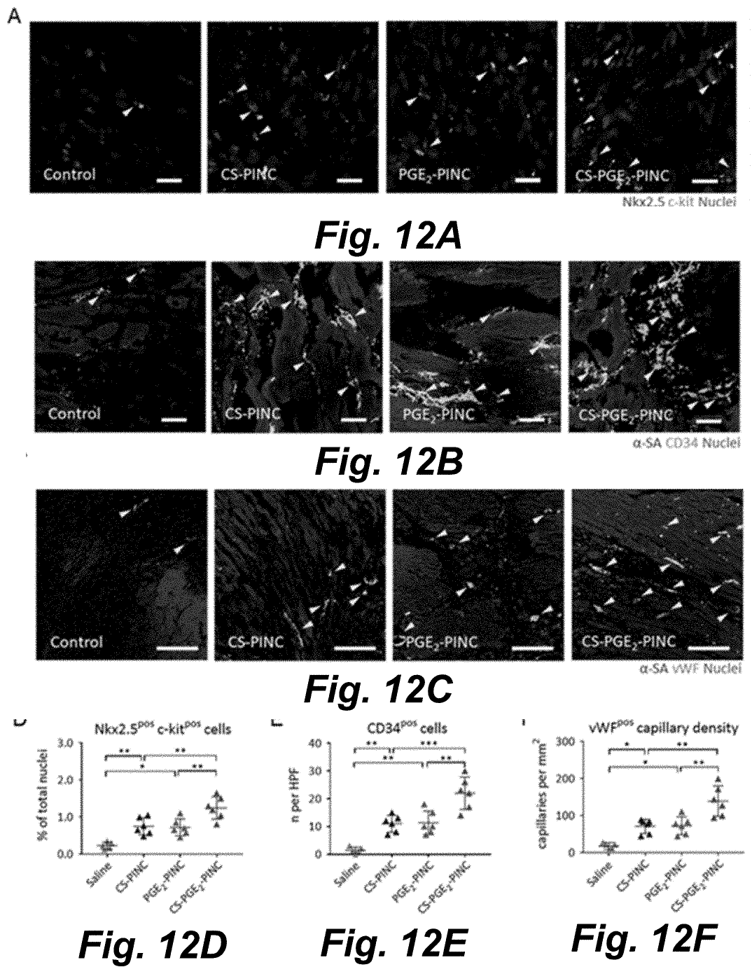

Functional Benefits of PINC Therapy in a Mouse Model of I/R: To test the potency of PINCs for the treatment of heart injury after I/R, adult mice were randomized to intravenously receive CS-PINCs, PGE₂-PINCs, CS-PGE₂-PINCs or saline injection after myocardial I/R injury. After 4 weeks of treatment, heart morphometry imaged by Masson's trichrome staining revealed the protective effects of PINC injections on heart morphology (FIG. 12A). When quantified, CS-PGE₂-PINC injection resulted in the highest amount of viable myocardium (FIG. 12B) with the smallest scar size (FIG. 12C). The reduced cardiac remodeling of CS-PGE₂-PINC-treated mice was further demonstrated by the reductions in left ventricular end diastolic volume (LVEDV) and end systolic volume (LVESV), respectively, compared with CS-PINCs, PGE₂-PINCs or saline controls (FIGS. 12D and 12E). Left ventricular ejection fraction (LVEF) were similar at baseline among all the groups, indicating a similar degree of initial injury. After 4 weeks, the LVEF of saline-treated animals evidently declined, while LVEF was well preserved in the CS-PINC and PGE₂-PINC treatment groups. The hearts of CS-PGE₂-PINC-treated animals showed the highest LVEF (FIG. 12F). From the treatment effects (i.e., change of LVEFs from baseline), saline injection had negative treatment effect; CS-PINCs and PGE₂-PINC treatments preserved cardiac functions, and CS-PGE₂-PINC injection robustly boosted cardiac functions (FIG. 12G).

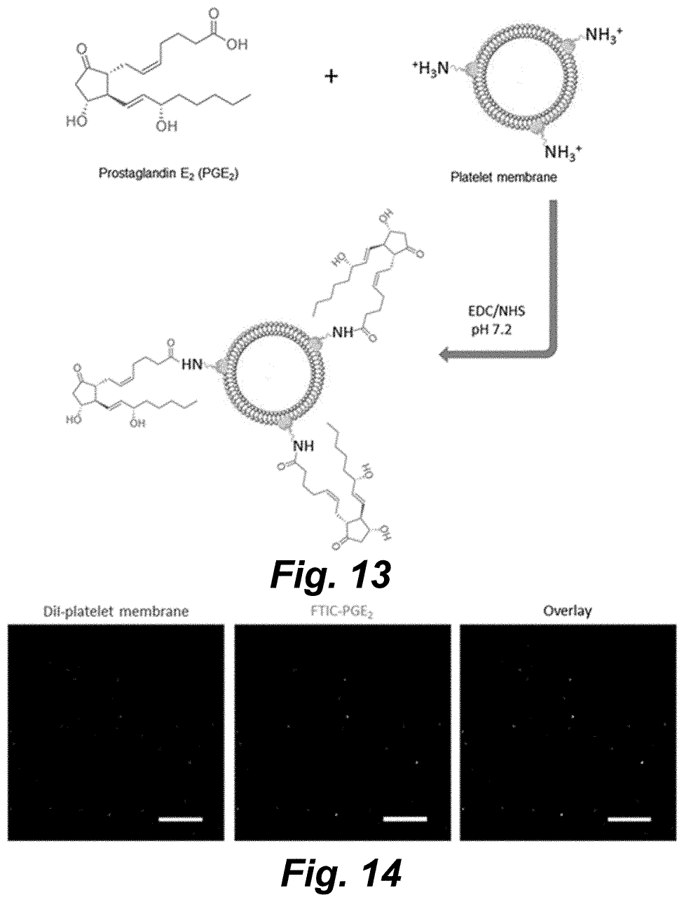

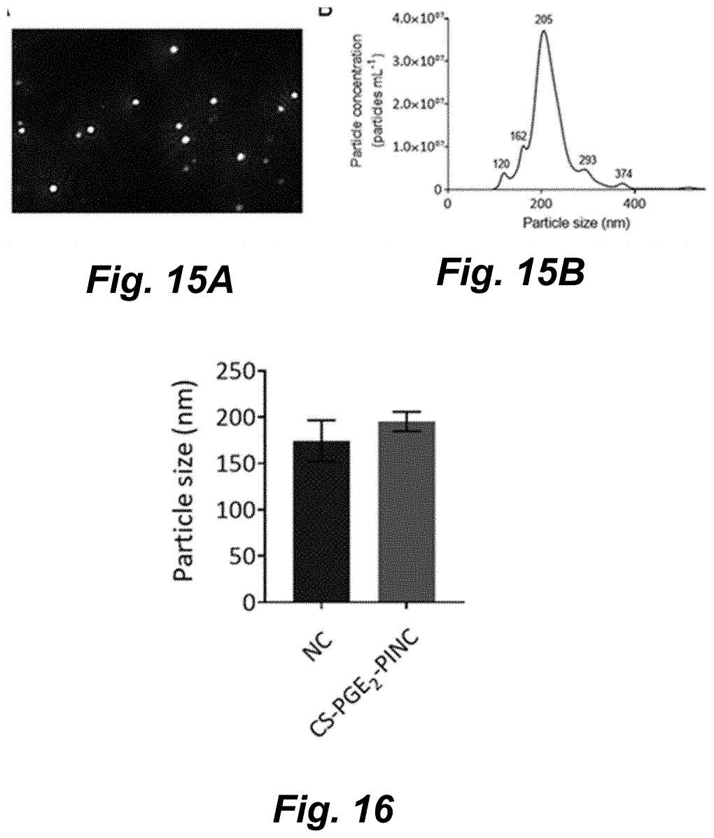

PINC Injection Promotes Endogenous Repair In the Infarcted Heart Immunostaining analysis was performed with c-kit nd Nkx2.5 (FIG. 13A), CD34 (FIG. 13B), and von Willibrand Factor (vWF, FIG. 13C) in the infarcted hearts of saline-, CS-PINC-, PEG₂-PINC-, and CS-PEG₂-PINC-treated mice. Compared with control, PINC treatments remarkably increased the recruitment of Nkx2.5- and c-kit-positive cardiac progenitor cells to the infarcted heart, with the highest number of cardiac progenitor cells entering the peri-infarct region of the CS-PGE₂-PINC-treated hearts (FIG. 13D). In addition, a greater number of CD34-positive cells were found in the CS-PGE₂-PINC-treated hearts compared with other treatment groups (FIG. 13E), suggesting the homing of endothelial stem/progenitor cells may also be elicited after PINC injection. This was corroborated by the enhanced vWF-positive vessel density found in the PINC-treated hearts compared with the control. Together, these results suggest that the therapeutic effects of PINCs may be through activation of Nkx2.5- and c-kit-positive cardiac progenitor cells and promotion of neovascularization.

To date, most nanoformulations aiming at treating myocardial infarction employed the passive targeting mechanism (i.e., enhanced permeability and retention (EPR) effect of the leaky vasculature in the acutely ischemic heart), thus were limited by the short-term retention from a few hours to days in the injured heart (Paulis et al., (2012) J. Control. Release 162: 276; Chang et al., (2013) J. Control. Release 170: 287). Christman, Gianneschi, and coworkers reported an enzyme-responsive nanoparticle that can form a network-like structure in response to the up-regulated MMPs following acute myocardial infarction for realizing long-term nanoparticle retention (Nguyen et al., (2015) Adv. Mater. 27: 5547).

The decoration of CSCs with platelet membrane nanovesicles boosts CSC retention in the infarcted myocardium and therapeutic outcomes in rats and pigs with ischemic heart injury (Tang et al., (2018) Nat. Biomed. Eng. 2: 17). In addition, it has been established that the expression levels of three subtypes of EPs, EP2, EP3, and EP4, are remarkably upregulated in the heart after myocardial I/R injury (Hishikari et al., (2009) Cardiovasc. Res. 81: 123; Calabresi et al., (2003) Circ. Res. 92: 330). PGE₂ can not only activate the endogenous stem/progenitor cells to replenish cardiomyocytes after ischemic injury through, but also enhance the recruitment and engraftment of CD34-positive hematopoietic stem cells after xenotransplantation through PGE₂/EP2/EP4 signaling (Hsueh et al., (2014) EMBO Mol. Med. 6: 496; Goessling et al., (2011) Cell Stem Cell 8: 445). The CS-PGE₂-PINCs reported here harness both the natural myocardial infarction-homing ability of platelet membrane and the upregulation of PGE₂ receptors in the cardiac microenvironment after I/R injury, resulting in prolonged retention in the infarcted heart after minimally-invasive intravenous delivery. Our findings showed that the released stem cell factors and PGE₂/EPs signaling could synergistically boost the therapeutic efficacy of CS-PGE₂-PINCs. As a result, the animals that received intravenous injection with CS-PGE₂-PINCs exhibited significantly augmented cardiac function and mitigated heart remodeling compared to other treatment groups. Such functional benefit was accompanied by the increase in cycling cardiomyocytes, activation of endogenous stem/progenitor cells, and promotion of angiogenesis.

The present disclosure, therefore, encompasses a novel platelet-inspired nano-cell that incorporates both PGE₂-modified platelet membrane and stem cell factors to target the heart after ischemic injury. CS-PGE₂-PINC was fabricated by functionalizing a CSC factor-releasing core with a platelet membrane shell with PGE₂ decoration. By taking advantage of the natural infarct-homing ability of platelet membrane and the overexpression of EPs in the pathological cardiac microenvironment, the CS-PGE₂-PINC can realize targeted delivery of the therapeutic payload to the injured heart. Furthermore, the synergetic treatment efficacy can be achieved by CS-PGE₂-PINC, which combines the paracrine mechanism of stem cell therapy with the PGE₂/EP receptor signaling that is involved in the repair and regeneration of multiple tissues (Zhang et al., (2015) Science 348: aaa2340; Goessling et al., (2011) Cell Stem Cell 8: 445; North et al., (2007) Nature 447: 1007). This platelet-inspired nanocell can be applied as a promising therapeutic delivery platform for treating multiple diseases.

One aspect of the disclosure encompasses embodiments of a stem cell biomimetic microparticle comprising a core nanoparticle and an outer layer disposed on the core nanoparticle, the core nanoparticle comprising at least one stem cell-derived secreted factor or a population of stem-cell derived exosomes embedded in a biocompatible polymer core nanoparticle, and wherein the outer layer is obtained from a cell membrane.

In some embodiments of this aspect of the disclosure, the stem cell can be a mesenchymal stem cell or a cardiac stem cell.

In some embodiments of this aspect of the disclosure, the cell membrane can be a red blood cell outer membrane or a stem cell outer membrane.

In some embodiments of this aspect of the disclosure, the cell membrane can be isolated from platelets.

In some embodiments of this aspect of the disclosure, the biocompatible polymer core nanoparticle can consist of a single species of polymer, a plurality of biocompatible polymer species, a block copolymer, or a plurality of polymer species, and wherein the polymer or polymers of the polymer core can be cross-linked.

In some embodiments of this aspect of the disclosure, the biocompatible polymer core nanoparticle can be biodegradable.

In some embodiments of this aspect of the disclosure, the biocompatible polymer core can comprise poly(lactic-co-glycolic acid) (PLGA).

In some embodiments of this aspect of the disclosure, the cell membrane can comprise a ligand attached thereto, wherein the ligand can specifically bind to a target cell or tissue.

In some embodiments of this aspect of the disclosure, the cell membrane can be isolated from platelets and the ligand is prostaglandin E2 (PGE₂).

In some embodiments of this aspect of the disclosure, the at least one stem cell-derived secreted factor or exosome population and the cell membrane layer can be from the same individual.

In some embodiments of this aspect of the disclosure, the at least one stem cell-derived secreted factor or exosome population and the cell membrane layer can be from different individuals.

In some embodiments of this aspect of the disclosure, the at least one stem cell-derived secreted factor can be from a stem cell-conditioned culture medium.

In some embodiments of this aspect of the disclosure, the at least one stem cell-derived secreted factor can be from a mesenchymal or cardiac stem cell-conditioned culture medium.

In some embodiments of this aspect of the disclosure, the at least one stem cell-derived secreted factor can be an isolated mesenchymal or cardiac stem cell-derived secreted factor.

In some embodiments of this aspect of the disclosure, the population of exosomes can be isolated from a medium conditioned by culturing therein a population of stem cells.

In some embodiments of this aspect of the disclosure, the population of exosomes can be isolated from a medium conditioned by culturing therein a population of stem cells.

In some embodiments of this aspect of the disclosure, the population of exosomes can be from a mesenchymal or cardiac stem cell-conditioned culture medium.

In some embodiments of this aspect of the disclosure, the microparticle can be suspended in a pharmaceutically acceptable carrier.

Another aspect of the disclosure encompasses embodiments of a method of generating a biomimetic microparticle, said method comprising the steps of: (a) admixing an aqueous solution comprising at least one stem cell-derived secreted factor or population of stem cell-derived exosomes with an organic phase having a polymerizable monomer dissolved therein; (b) emulsifying the admixture from step (a); (c) admixing the emulsion from step (b) with an aqueous solution of polyvinyl alcohol and allowing the organic phase to evaporate, thereby generating polymer nanoparticles having the at least one stem cell-derived secreted factor embedded therein; (d) obtaining an isolated cell membrane or fragments thereof; and (e) generating cell membrane-coated biomimetic microparticles by mixing the polymer nanoparticles from step (c) with the suspension of isolated red blood cell membrane or fragments thereof.

In some embodiments of this aspect of the disclosure, the aqueous solution comprising the at least one stem cell-derived secreted factor can be a stem cell-conditioned culture medium or an aqueous solution of at least one isolated stem cell secreted factor.

In some embodiments of this aspect of the disclosure, the aqueous solution can be a mesenchymal stem cell- or a cardiac stem cell-conditioned culture medium.

In some embodiments of this aspect of the disclosure, the first polymerizable monomer polymer can be poly(lactic-co-glycolic acid) (PLGA).

In some embodiments of this aspect of the disclosure, the step (c) can further comprise lyophilizing the polymer microparticles.

Yet another aspect of the disclosure encompasses embodiments of a method of treating a pathological condition of a patient by delivering to the patient in need thereof a pharmaceutically acceptable composition comprising a stem cell biomimetic microparticle, wherein the stem cell biomimetic microparticle comprises at least one stem cell-derived secreted factor or population of stem cell-derived exosomes embedded in a biocompatible polymer core microparticle and an outer layer derived from red blood cell membranes or platelet membranes disposed on the biocompatible polymer core microparticle.

In some embodiments of this aspect of the disclosure, the pathological condition of the patient can be a disease of the liver.

In some embodiments of this aspect of the disclosure, the pathological condition of the patient can be a cardiovascular disease or injury.

In some embodiments of this aspect of the disclosure, the biocompatible polymer core of the microparticle can consist of a single species of polymer, a plurality of polymer species, a block copolymer, or a plurality of polymer species, and wherein the polymers of the polymer core can be cross-linked.

In some embodiments of this aspect of the disclosure, the polymer core of the microparticle can be biodegradable.

In some embodiments of this aspect of the disclosure, the polymer core can comprise poly(lactic-co-glycolic acid) (PLGA).

In some embodiments of this aspect of the disclosure, the at least one stem cell-derived secreted factor and the cell membrane fragment or fragments can be from the same individual.

In some embodiments of this aspect of the disclosure, the at least one stem cell-derived secreted factor and the cell membrane fragment or fragments can be from different individuals.

In some embodiments of this aspect of the disclosure, the at least one stem cell-derived secreted factor can be from a mesenchymal stem cell- or cardiac stem-cell-conditioned culture medium.

In some embodiments of this aspect of the disclosure, the at least one stem cell-derived secreted factor can be an isolated secreted factor.

In some embodiments of this aspect of the disclosure, the pharmaceutically acceptable composition can be formulated for delivery directly into a target tissue of a subject animal or human or for local or systemic administration to a subject animal or human.

It should be emphasized that the embodiments of the present disclosure, particularly any “advantageous” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following claims.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more.

### EXAMPLES

### Example 1

Preparation of MSC conditioned medium-loaded PLGA nanoparticles (NP): Human bone marrow-derived MSC was directly obtained from ATCC (Cat #: 63208778). The cells were cultured per vendor's instructions. The MSCs were cultured in Iscove's Modified Dulbecco's Medium (IMDM, Thermo Fisher Scientific) for 3 days and then the supernatant was collected to harvest conditioned media. Conditioned media was collected and filtered through a 0.22 μM filter into a sterile 50 mL conical to remove any cell debris and contaminants. Sterile conditioned media was stored at −80° C. for at least 24 hours then lyophilized by a freeze-dry system. MSC conditioned media-loaded PLGA NPs were fabricated by a double emulsion process followed by membrane extrusion (Zhai et al., (2017) Theranostics 7: 2575-2592). In brief, concentrated human MSC conditioned media was prepared as the internal aqueous phase in polyvinyl alcohol (PVA) (0.1%) and injected into dichloromethane (DCM) containing polylactic-co-glycolic acid (PLGA). The whole content was then sonicated on ice for emulsification. Afterward, the emulsion was immediately transferred into water with PVA. The secondary emulsion was emulsified again to produce the final water/oil/water mixture. Then, it was stirred overnight to promote solvent evaporation. To enable detection, PLGA was pre-labeled with Cy5.5 or AF594 fluorophore. The solidified nanoparticles were then centrifuged and washed with water. The particles were then extruded through membranes with pore sizes of 400 nm and 200 nm using an extruder (Avanti Polar Lipids, Alabaster, Ala.).

### Example 2

Flow cytometry: To characterize the phenotypes of MSC, flow cytometry was performed using a Beckman Coulter flow cytometer (Brea, Calif.). Primary antibodies used are: CD31

(BD555445), CD34 (BD555821), CD45 (BD 555482), CD90 (BD 555595) and CD105 (FAB 10971P, R&D Systems) from BD company or R&D Systems.

### Example 3

Generation of RBC membrane vesicles: The whole blood withdraws from C57BL/6 mice (6 weeks, 18-25 g, Charles River Laboratories) was centrifuged at 800 g to obtain RBC pellets. The RBCs were then washed 3 times with cold PBS. After that, hemolysis was triggered by treatment with a hypotonic solution and subsequently centrifuged at 800 g for 5 min. The resulting RBC shells (free of cytoplasmic components) were washed with PBS and examined using a white light microscope (Gao et al., (2013) Adv. Mater. 25: 3549-3553). To prepare RBC membrane vesicles, RBC shells were subjected to three freeze/thaw cycles. After that, the collected RBC shells were extruded through membranes with pore sizes of 400 nm and 200 nm using an extruder (Avanti Polar Lipids).

### Example 4

Fabrication and characterization of MR/N: To cloak the RBC vesicles onto the surface of MSC-NPs, 0.5 mL of MSC-NPs (5×10⁹/ml) was mixed with 0.5 ml RBC vesicles (5×10⁹/ml) and then extruded 11 times as previously described. The resulting MRINs were centrifuged at 800×g to remove excessive membrane debris. Nanoparticle concentration and size were examined by NanoSight (Malvern, UK). Surface charge (zeta potential, mV) were measured by dynamic light scattering (DLS). The morphology of MRINs was studied by TEM (JEOL JEM-2000FX). The coated specimen was imaged after negative staining with 1 wt % uranyl acetate. To reveal whether MRINs have the same signature proteins as RBCs, SDS-PAGE was performed to reveal the protein components of RBC membrane vesicles and the dialyzed MRIN particles.

### Example 5

Growth factors release study and pharmacokinetic studies in vivo: Total protein and growth factor releases from MRINs were determined as previously described (Luo et al., (2017) Circ. Res. 120: 1768-1775; Tang et al., (2017) Nat Commun. 8: 13724). In brief, freeze-dried MRI Ns were dissolved in DCM. After that, PBS was added to the solution. The sample was subjected to vortex for 5-10 min to isolate proteins from the oil phase to the water phase. After centrifugation, the protein concentration in the water phase was measured by a bicinchoninic acid assay. For growth factor release studies, nanoparticles were incubated in PBS at 37° C. The supernatant was collected at different time points (24, 48, 72, 96, 120,144 hrs) and the concentrations of various growth factors were determined by enzyme-linked immunosorbent assays (ELISAs). 10 male C57BL/6 mice were randomized into 2 groups (n=5 per group) and were intravenously injected with NP and MRIN nanoparticles (both labeled with Cy5.5 fluorophore during nanoparticle preparation). At 0.5, 1, 2, 4, 12, 24, 48 hrs, 20 μl whole blood was collected. Nanoparticle concentrations were determined by UV— vis spectra using a Nanodrop 2000 (Thermo Scientific, USA). Known concentrations of Cy5.5-labeled NPs were mixed with blood to generate a standard calibration curve. The concentrations of NPs in different blood samples were calculated based on the standard curve.

### Example 6

Macrophage uptake study: Human mononuclear cells (hMNCs) were isolated from healthy volunteers. A Ficoll separation method was followed. CD14+ cells were purified by magnetic-activated cell sorting (MACS, Miltenyi Biotec) and differentiated to macrophages, using RPMI media supplemented with 1% glutamine and 10% human serum (Pallotta et al., (2017) J. Tissue Eng. Regen. Med. 11: 1466-1478). M1 macrophages were enriched with further polarization. At the 7th day, M1 macrophages were co-incubated with RBC-coated MRINs or uncoated control NPs at 37° C. for 3 hrs and then washed with PBS for 3 times for imaging. For imaging purposes, both MRINs and NPs were pre-labeled with AF594 fluorophore. After the co-incubation period, cells were fixed with 4% glutaraldehyde at room temperature, counterstained with DAPI before microscopic imaging.

### Example 7

In vitro cell-based assays: The immortalized liver cell line (THLE-2) was obtained from ATCC (CRL-2706,) and cultured in a BEGM Bullet Kit (Lonza, CC3170) supplementation with 70 ng/mL phosphoethanolamine (Sigma, P0503), 5 ng/mL EGF (Sigma, E5036), and 10% fetal calf serum superior (sigma). HSC-T6 cell line was obtained from Sigma (SCC069) and cultured in IMDM media (Invitrogen, Carlsbad, Calif.) with 10% FBS (Corning), 0.5% Gentamicin, 0.1 mM 2-mercaptoethanol, and 1% L-glutamine. Primary mouse lung cells were obtained and maintained with the lung spheroid methods as previously described (Cores et al., (2017) Stem Cells Transl. Med. 6: 1905-1916; Dinh et al., (2017) Respir. Res. 18: 132; Henry et al., (2015) Stem Cells Transl. Med. 4: 1265-1274). To mimic liver injury, all cells were incubated with 20 mmol/ml CCL4 for 24 hrs. After that, the cells were further incubated with different concentration of MRINs for 24 hrs. A Cell Counting Kit-8 (CCK-8, 96992 Sigma) was used to evaluate cell proliferation.

### Example 8

Ex vivo fluorescent imaging for biodistribution of MRINs: Cohorts of normal mice and liver failure mice were scarified 6, 12, and 24 hr after nanoparticle injections; major organs were collected for biodistribution studies using ex vivo fluorescent imaging (IVIS, Caliper Lifesciences, Waltham, Mass.).

### Example 9

Mouse model of acute liver failure and MRIN therapy: All animal work was compliant with the IACUC at North Carolina State University. Briefly, C57BL/6 mice (body weight 18-25 g) were anesthetized with isofluorane inhalation. Then, 4.0 ml/kg CCl4 (Sigma, 2891116) mixed with olive oil (Sigma, 01514) (1:1 ratio) was injected intraperitoneally. After 24 hrs, the liver failure model was confirmed by histology. After that, the mice were randomized into the following treatment arms: (1) Sham group: No liver failure induction or therapy; (2) PBS Control group: Liver failure+tail vein injection of 200 μl PBS to the liver failure mice; (3) CM group: liver failure+tail vein injection of 1 mg CM lyophilized powder dissolved in 200 μl PBS; (4) NP group: liver failure+tail vein injection of 1×10⁹ NPs in 200 μl PBS; (5) MRIN group: tail injection of 1×10⁹ MRINs in 200 μl. The mice were given the injections twice a week for two weeks (total 4 injections).

### Example 10

Liver/kidney function test and cytokine assay: Liver and kidney functions were examined by measuring serum ALT and AST levels, and blood urea nitrogen (BUN), creatinine (Cr) by the veterinary hospital of NC State University. The serum levels of IL-1β (Sigma, RAB0275), IL-6 (Sigma, RAB0308) and TNF-α (Sigma, RAB0477) were measured with commercially available ELISAs. Survival rates were analyzed using a Kaplan-Meier plot with log-rank analysis.

### Example 11

Histology: Liver tissues were kept in 10% formalin and embedded in paraffin. After cutting, liver sections (5 um thick) were subjected to hematoxylin and eosin (H&E) staining. Images were taken by a microscopy (AZ-100). For immunohistochemistry, 10 um-thick liver cryo-sections were fixed with 4% paraformaldehyde (PFA) and permeabilized/blocked. The slides were then incubated with rabbit anti-Ki67 (1:100, ab15580, Abcam) overnight at 4° C. After that the cells were incubated with Texas-Red secondary antibodies (1:100, ab6719, Abcam) and counterstained with DAPI. For assessment of cell apoptosis, liver cryo-sections were incubated with TUNEL solution (Roche Diagnostics, Germany) and counterstained with DAPI. To study the cellular location of MRINs in the liver, liver cryo-sections were incubated with anti-WGA (1:50, ab20528, abcam) for liver cells, anti-CD45(1:200, ab10558, abcam) for macrophages, followed by incubation with AF488 secondary antibodies (1:200, ab150077, abcam). Images were taken by a confocal microscope (Carl Zeiss, Germany).

### Example 12

Statistical analysis: All data were presented as the mean±SD. Two-tailed Student's t-test was used for comparison between 2 groups. Comparisons among 3 or more groups were performed using one-way ANOVA. Statistical significance was considered when P<0.05.

### Example 13

Preparation of CSC Secretome-loaded PLGA Nanoparticles (NCs): Human CSCs were derived from donor human hearts according to Tang et al., ((2018) Nat. Biomed. Eng. 2: 17). All procedures were approved by the institutional review board and written informed consent was obtained from all patients. The CSCs were used at passages 2-4. Briefly, the CSCs were cultured in serum-free media for 3 days and then the supernatant was collected to harvest conditioned media. The collected conditioned media was filtered through a 0.22 μM filter into a sterile 50 mL conical to remove any cell debris and contaminants. Sterile conditioned media was then lyophilized by a LABCONCO FreeZone 2.5 Liter Freeze Dry System to produce the purified CSC secretome. The CSC secretome-loaded PLGA cores were fabricated through a double emulsion method followed by membrane extrusion. In brief, concentrated CSC secretome aqueous solution was prepared as the internal aqueous phase in polyvinyl alcohol (PVA, MW: 30000-50000, 0.1% w/v), and then injected into dichloromethane (DCM) containing PLGA (MW: 7000-17000) as the oil phase. The whole content was sonicated on ice for emulsification using a Misonix XL2020 sonicator. Afterward, the emulsion was immediately transferred into water containing 0.7% (w/v) PVA. The secondary emulsion was emulsified again to produce the final water/oil/water PLGA nanoparticle emulsion. The w/o/w emulsion was continuously stirred overnight to promote solvent evaporation. The nanoparticles were then sequentially extruded 19 times through polycarbonate membranes with pore sizes of 400 and 200 nm, respectively, using an extruder (Avanti Polar Lipids, USA). To determine the secretome loading capacity and efficiency, the nanoparticles were pelleted by centrifugation at 20000×g and then the non-encapsulated amount of secretome in the supernatant was measured using a bicinchoninic acid (BCA) protein assay.

### Example 14

Isolation of Platelet Membrane: The platelet membrane was isolated from human platelet-rich plasma (PRP, ZenBio, USA) through gradient centrifugation (Tang et al., (2018) Nat. Biomed. Eng. 2: 17). The PRP was centrifuged at 100×g for 20 mins. PBS (1×, pH 7.4) containing 1 mM of ethylenediaminetetraacetic acid and 2 μM of prostaglandin E1 was added to keep platelets inactivated. The isolated platelets were then pelleted by centrifugation at 800×g for 20 mins at room temperature. The platelet membrane was obtained by three freeze-thaw cycles, followed by sonication. The protein content in the purified platelet membrane was determined by BCA protein assay for further preparation of PINCs.

### Example 15

Preparation of PGE₂-Platelet Membrane Conjugate: PG E2-platelet membrane conjugate was synthesized using EDC/NHS coupling chemistry. PGE₂(0.52 μg, 1.5 nmol) dissolved in anhydrous ethanol was reacted with EDC (58 μg, 0.37 μmol) and NHS (14 μg, 0.12 μmol) in 15 mL 2-(4-Morpholino) ethane sulfonic acid (MES) buffer (100 mM, pH 6.0). After 30 min of carboxylate activation, platelet membrane containing 0.36 mg protein was added. The pH of the reaction mixture was adjusted to 7.2 by the addition of 1 M sodium bicarbonate. The mixture was stirred overnight at room temperature. The resulting product was dialyzed (MWCO 1000) for 24 h against PBS. The amount of unconjugated PGE₂ was measured by ELISA (R&D Systems, USA) to determine the conjugation yield and the content of PGE₂ conjugated to the PGE2-platelet membrane conjugate.

### Example 16

Fabrication and Characterization of PINCs: To cloak the platelet membrane or PGE₂-platelet membrane conjugate onto the surface of CSC secretome-loaded NCs, 0.5 mL of NCs (5×10⁹ particles mL⁻1) were incubate with 0.5 mL of platelet membrane or PGE₂-platelet membrane conjugate containing 0.36 mg membrane protein under ultrasonication for 5 min, and then extruded 19 times as previously described to prepare the PINCs. The CS-PGE₂-PINCs were prepared via coating the PGE₂-platelet membrane conjugate on the surface of NCs. The CS-PINCs were prepared via coating the platelet membrane on the surface of NCs while the PGE₂-PINCs were prepared via coating the PGE₂-platelet membrane conjugate on the surface of bare PLGA nanoparticle.

### Example 17

Physicochemical Characterization: Nanoparticle size and surface zeta potential were measured by dynamic light scattering (DLS) using a Malvern ZEN 3600 Zetasizer. Nanoparticle concentration was examined by NanoSight NS300. The nanoparticle structure was visualized using a JEOL JEM-2000FX transmission electron microscope after negative staining with vanadium (Abcam). To assess the stability of the different nanoformulations over time, the bare NCs, CS-PINCs, PGE₂-PINCs, and CS-PGE₂-PINCs were suspended in PBS (1×, pH 7.4) at a concentration of 10⁹ particles mL⁻¹. The change of particle size was measured by DLS at pre-determined time-points over the course of 2 weeks. To evaluate the stability in serum, the different nanoformulations were incubated with 50% fetal bovine serum (Hyclone, USA). The change of particle sizes within 4 h was determined by DLS. Long-term stability was assessed by the particle size change measured by DLS before lyophilization in 10 wt % sucrose and after resuspension in PBS (1×, pH 7.4) back to the original volume. SDS-PAGE was performed to examine the protein components of platelet membrane and the different PINCs.

### Example 18

Collagen Surface Binding Assay: GFP-tagged HUVECs (Angio-Proteomie, USA) were seeded on collagen-coated 4-well culture chamber slides and cultured for 24 h. The cells were then incubated with Dil-labeled CS-PGE₂-PINCs in PBS (1×, pH 7.4) at 4° C. for 60 s. Then the cells were washed with PBS twice. Images were taken on an Olympus IX81 fluorescent microscope and analyzed using NIH ImageJ software.

### Example 19

Growth Factor Release Study: Total protein and growth factor releases from PINCs were determined (Luo et al., (2017) Circ. Res. 120: 1768; Tang et al., (2017) Nat. Commun. 8: 13724). Freeze-dried PINCs were dissolved in DCM. After that, PBS was added to the solution. The sample was subjected to vortex for 5 min to isolate proteins from the oil phase to the water phase. After centrifugation, the protein concentration in the water phase was measured by BCA protein assay. For growth factor release studies, nanoparticles were incubated in PBS at 37° C. The supernatant was collected at various time points (day 3, 7, 11, 14) after centrifugation at 20000×g for 30 min to pellet the nanoparticles. The concentrations of various growth factors were measured using ELISA kits (R&D Systems, USA) according to the manufacturer's instructions. The data were averaged from three independent measurements.

### Example 20

Cell Viability and Proliferation Assay: The H9c2 cardiomyoblasts derived from embryonic rat heart (Sigma-Aldrich) were incubated with different concentrations of PINCs. PBS (1×, pH 7.4) was used as a control. The cell viability and proliferation were assessed by using a Cell Counting Kit-8 (CCK-8) according to the manufacturer's instructions.

### Example 21

NRCM Uptake Assay: NRCMs were derived from SD rats and cultured on 4-well chamber slides for 3 days, followed by co-incubation with Dil-labeled CS-PINCs or CS-PGE₂-PINCs (1.5×10⁹ particles mL⁻1) in an incubator for 3 h. The beating cardiomyocytes were counted under a white-light microscope. Then, the cells were washed with PBS twice, fixed, permeabilized, and stained for α-sarcomeric actinin (α-SA, 1:200, ab9465, Abcam), followed by DAPI staining for nucleus visualization. For assessment of cell apoptosis, the cells were cocultured with CS-PINCs or CS-PGE₂-PINCs (1.5×10⁹ particles mL⁻1) for 48 h. Subsequently, the cells were washed with PBS twice and then exposed to serum-free medium supplemented with hydrogen peroxide (250 μM) for 3 h, followed by incubated with TUNEL solution (Roche Diagnostics GmbH, Germany) and counter-stained with DAPI. Images were taken using a Zeiss LSM 710 confocal microscope (Carl Zeiss, Germany) and analyzed using NIH ImageJ software.

### Example 22

Mouse Model of Myocardial I/R Injury: All animal work was compliant with the Institutional Animal Care and Use Committee (IACUC) of University of North Carolina at Chapel Hill and North Carolina State University. Briefly, female immunocompetent CD1 mice (8-10 weeks old, Charles River Laboratories) were anesthetized by inhalation of 3% isoflurane in 100% oxygen at a flow rate of 2 L·min⁻¹. Under sterile condition, the heart was exposed by a left thoracotomy and a 30-min ischemia was achieved by temporal ligation of the left anterior descending coronary artery. After 24 h of reperfusion, animals were randomized to receive intravenous injection of CS-PINCs (CS dose: 1.2 mg kg⁻¹ mouse), PGE₂-PINCs (PGE₂ dose: 0.053 mg kg⁻mouse), CS-PGE₂-PINCs (CS dose: 1.2 mg kg⁻¹ mouse, PGE₂ dose: 0.053 mg kg⁻¹ mouse), and saline (control) every 7 days for 4 weeks.

### Example 23

In Vivo Targeting Ability Study: Immunocompetent CD1 mice with myocardial I/R injury were randomized into 2 groups (n=3 per group) to receive intravenous injections with DiR-labeled NCs or CS-PGE₂-PINCs (CS dose: 1.2 mg kg⁻¹ mouse, PGE₂ dose: 0.053 mg kg⁻¹ mouse) every 7 days for 2 weeks. After that, the mice were euthanized and the major organs (heart, lung, liver, kidney, spleen) were carefully collected. The fluorescence signals of each organ were recorded using the IVIS Spectrum imaging system for quantification.

### Example 24

Cardiac Function Assessment: All animals underwent transthoracic echocardiography under anesthesia at 4 h post-I/R injury and 4 weeks after treatment using a VisualSonics Vevo 2100 Imaging System. Hearts were imaged two-dimensionally in long-axis views at the level of the greatest left ventricular diameter. Left ventricular end-diastolic volume (LVEDV) and end-systolic volume (LVESV) were measured. Left ventricular ejection fraction (LVEF) was determined by measurement from views taken from the infarcted area. All measurements were done in random order, with the surgeon and echocardiographer being blind to the treatment groups.

### Example 25

Heart Morphometry: Hearts were harvested and cut into 10 μm-thick tissue sections. Masson's trichrome staining was performed and images were acquired with a PathScan Enabler IV slide scanner (Advanced Imaging Concepts, USA). Image analysis related to viable myocardium and scar size was performed using NIH ImageJ software. Three selected sections were quantified for each animal.

### Example 26

Immunohistochemistry assessment: Heart cryosections were fixed with 4% paraformaldehyde in PBS for 30 min, permeabilized and blocked with Protein Block Solution (DAKO) containing 0.1% saponin for 1 h at room temperature. For immunostaining, the samples were incubated overnight at 4° C. with the following primary antibodies diluted in the blocking solution: rabbit anti-mouse α-SA (1:200, ab137346, Abcam) was used to identify cardiomyocytes; rat anti-mouse Ki67 antibody (1:200, 151202, Biolegend), rabbit anti-mouse histone H3 phosphorylated at serine 10 (pH 3, 1:200, ab5176, Abcam), and rabbit anti-mouse Aurora B kinase (AURKB, 1:200, ab2254, Abcam) antibodies were used to analyse cell-cycle re-entry, karyokinesis, and cytokinesis, respectively; sheep anti-mouse vWF (1:200, ab11713, Abcam) antibody was used to detect myocardial capillaries in the peri-infarct regions; goat anti-mouse Nkx2.5 (1:200, ab106923, Abcam), rabbit anti-mouse c-kit (1:200, ab62154, Abcam), and rat anti-mouse CD34 (1:200, MA1-22646, Thermo Fisher Scientific) antibodies were used to examine endogenous progenitor/stem cell recruitment; rabbit anti-mouse CD3 (1:200, ab16669, Abcam), rat anti-mouse CD8 (1:200, ab22378, Abcam), and rabbit anti-mouse CD68 (1:200, ab125212, Abcam) antibodies were used to detect immune response. After three 10-min washes with PBS, samples were stained for 1.5 h at room temperature with fluorescent secondary antibodies including goat anti-rabbit IgG-Alexa Fluor 594 conjugate (1:400, ab150080, Abcam), goat anti-rat IgG-Alexa Fluor 488 conjugate (1:400, ab150157, Abcam), donkey anti-rabbit IgG-Alexa Fluor 488 conjugate (1:400, ab150073, Abcam), donkey anti-goat IgG-Alexa Fluor 594 conjugate (1:400, ab150136, Abcam), donkey anti-sheep IgG-Alexa Fluor 488 conjugate (1:400, ab150177, Abcam), goat anti-rabbit IgG-Alexa Fluor 488 conjugate (1:400, ab150077, Abcam), and goat anti-rabbit IgG-Alexa Fluor 594 conjugate (1:400, ab150080, Abcam), and goat anti-rat IgG-Cy5 conjugate (1:400, ab6563, Abcam) based on the isotopes of the primary antibodies. This was followed by 10 min of 4, 6-diamidino-2-phenylindole dihydrochloride (DAPI) staining for nucleus visualization. Slides were mounted with ProLong Gold mountant (Thermo Fisher Scientific) and viewed under a Zeiss LSM 710 confocal microscope (Carl Zeiss). Images were analyzed using NIH ImageJ software.

We claim:

1. A stem cell biomimetic microparticle comprising a core nanoparticle and an outer layer disposed on the core nanoparticle, the core nanoparticle comprising at least one stem cell-derived secreted factor or a population of stem-cell derived exosomes embedded in a biocompatible polymer core nanoparticle, and wherein the outer layer is obtained from a cell membrane.

2. The stem cell biomimetic microparticle of claim 1, wherein the stem cell is a mesenchymal stem cell or a cardiac stem cell.

3. The stem cell biomimetic microparticle of claim 1, wherein the cell membrane is a red blood cell outer membrane or a stem cell outer membrane.

4. The stem cell biomimetic microparticle of claim 1, wherein the cell membrane is isolated from platelets.

5. The stem cell biomimetic microparticle of claim 1, wherein the biocompatible polymer core nanoparticle consists of a single species of polymer, a plurality of biocompatible polymer species, a block copolymer, or a plurality of polymer species, and wherein the polymer or polymers of the polymer core are optionally cross-linked.

6. The stem cell biomimetic microparticle of claim 2, wherein the biocompatible polymer core nanoparticle is biodegradable.

7. The stem cell biomimetic microparticle of claim 1, wherein the biocompatible polymer core comprises poly(lactic-co-glycolic acid) (PLGA).

8. The stem cell biomimetic microparticle of claim 1, wherein the cell membrane comprises a ligand attached thereto, wherein the ligand specifically binds to a target cell or tissue.

9. The stem cell biomimetic microparticle of claim 8, wherein the cell membrane is isolated from platelets and the ligand is PGE₂.

10. The stem cell biomimetic microparticle of claim 1, wherein the at least one stem cell-derived secreted factor or exosome population and the cell membrane layer are from the same individual.

11. The stem cell biomimetic microparticle of claim 1, wherein the at least one stem cell-derived secreted factor or exosome population and the cell membrane layer are from different individuals.

12. The stem cell biomimetic microparticle of claim 1, wherein the at least one stem cell-derived secreted factor is from a stem cell-conditioned culture medium.

13. The stem cell biomimetic microparticle of claim 1, wherein the at least one stem cell-derived secreted factor is from a mesenchymal or cardiac stem cell-conditioned culture medium.

14. The stem cell biomimetic microparticle of claim 1, wherein the at least one stem cell-derived secreted factor is an isolated mesenchymal or cardiac stem cell-derived secreted factor.

15. The stem cell biomimetic microparticle of claim 1, wherein the population of exosomes are isolated from a medium conditioned by culturing therein a population of stem cells.

16. The stem cell biomimetic microparticle of claim 15, wherein the population of exosomes are isolated from a medium conditioned by culturing therein a population of stem cells.

17. The stem cell biomimetic microparticle of claim 1, wherein the population of exosomes is from a mesenchymal or cardiac stem cell-conditioned culture medium.

18. The stem cell biomimetic microparticle of claim 1, wherein the microparticle is suspended in a pharmaceutically acceptable carrier.

19. A method of generating a biomimetic microparticle, said method comprising the steps of:
(a) admixing an aqueous solution comprising at least one stem cell-derived secreted factor or population of stem cell-derived exosomes with an organic phase having a polymerizable monomer dissolved therein;
(b) emulsifying the admixture from step (a);
(c) admixing the emulsion from step (b) with an aqueous solution of polyvinyl alcohol and allowing the organic phase to evaporate, thereby generating polymer nanoparticles having the at least one stem cell-derived secreted factor embedded therein;
(d) obtaining an isolated cell membrane or fragments thereof; and
(e) generating cell membrane-coated biomimetic microparticles by mixing the polymer nanoparticles from step (c) with the suspension of isolated red blood cell membrane or fragments thereof.

20. The method of step 19, wherein the aqueous solution comprising the at least one stem cell-derived secreted factor is a stem cell-conditioned culture medium or an aqueous solution of at least one isolated stem cell secreted factor.

21. The method of step 19, wherein the aqueous solution is a mesenchymal stem cell- or a cardiac stem cell-conditioned culture medium.

22. The method of step 19, wherein the first polymerizable monomer polymer is poly(lactic-co-glycolic acid) (PLGA).

23. The method of step 19, wherein step (c) further comprises lyophilizing the polymer microparticles.

24. A method of treating a pathological condition of a patient by delivering to the patient in need thereof a pharmaceutically acceptable composition comprising a stem cell biomimetic microparticle, wherein the stem cell biomimetic microparticle comprises at least one stem cell-derived secreted factor or population of stem cell-derived exosomes embedded in a biocompatible polymer core microparticle and an outer layer derived from red blood cell membranes or platelet membranes disposed on the biocompatible polymer core microparticle.

25. The method of claim 24, wherein the pathological condition of the patient is a disease of the liver.

26. The method of claim 24, wherein the pathological condition of the patient is a cardiovascular disease or injury.

27. The method of claim 24, wherein the biocompatible polymer core of the microparticle is consists of a single species of polymer, a plurality of polymer species, a block copolymer, or a plurality of polymer species, and wherein the polymers of the polymer core are optionally cross-linked.

28. The method of claim 24, wherein the polymer core of the microparticle is biodegradable.

29. The method of claim 24, wherein the polymer core comprises poly(lactic-co-glycolic acid) (PLGA).

30. The method of claim 24, wherein the at least one stem cell-derived secreted factor and the cell membrane fragment or fragments are from the same individual.

31. The method of claim 24, wherein the at least one stem cell-derived secreted factor and the cell membrane fragment or fragments are from different individuals.

32. The method of claim 24, wherein the at least one stem cell-derived secreted factor is from a mesenchymal stem cell- or cardiac stem-cell-conditioned culture medium.

33. The method of claim 24, wherein the at least one stem cell-derived secreted factor is an isolated secreted factor.

34. The method of claim 24, wherein the pharmaceutically acceptable composition is formulated for delivery directly into a target tissue of a subject animal or human or for local or systemic administration to a subject animal or human.
